(19) United States
(12) Patent Application Publication
(10) Pub. No.: US 2024/0269133 A1
(43) Pub. Date: Aug. 15, 2024

(54) METHODS OF TREATING INTERSTITIAL CYSTITIS/BLADDER PAIN SYNDROME

(71) Applicant: Purdue Pharma L.P., Stamford, CT (US)

(72) Inventors: Garth WHITESIDE, Yardley, PA (US); Stephen HARRIS, Weston, CT (US)

(73) Assignee: Purdue Pharma L.P., Stamford, CT (US)

(21) Appl. No.: 18/561,405

(22) PCT Filed: May 20, 2022
(86) PCT No.: PCT/US2022/030400

§ 371 Date: Nov. 16, 2023

Related U.S. Application Data
(60) Provisional application No. 63/191,536, filed on May 21, 2021.

(51) Int. Cl. A61K 31/498; A61K 31/137; A61K 31/216; A61K 31/4025; A61K 31/46; A61K 31/4725; A61P 13/10

(52) U.S. Cl. CPC A61K 31/498; A61K 31/137; A61K 31/216; A61K 31/4025; A61K 31/46; A61K 31/4725; A61P 13/10

(57) ABSTRACT

The disclosure provides a method of treating interstitial cystitis/bladder pain syndrome in a human subject in need of such treatment, comprising administering to the human subject a therapeutically effective amount of a compound of formula (I) or a pharmaceutically acceptable salt thereof. In certain embodiments, the method comprises administering the compound of formula (IA).

### BACKGROUND

Interstitial cystitis/bladder pain syndrome is a debilitating chronic disease characterized by suprapubic pain related to bladder filling, coupled with additional symptoms such as increased day- and night-time urinary frequency, urgency, nocturia, and pelvic discomfort. See Yoshimura et al. (2014), Int J UroL 2014, April; 21 Suppl 1(01): 18-25. Patients suffering from interstitial cystitis/bladder pain syndrome experience a breakdown of the glycosaminoglycan layer that protects the bladder epithelium, which results in irritation of the bladder wall. Patients suffering from interstitial cystitis/bladder pain syndrome can experience moderate or severe pain.

Medications such as antihistamines, antidepressants and anticonvulsants have been used to treat interstitial cystitis/bladder pain syndrome, but with limited success. The drug pentosanpolysulfate sodium, which restores a damaged or leaky bladder surface, has been approved for the treatment of interstitial cystitis/bladder pain syndrome. However, most patients do not effectively respond to this medication.

While the exact mechanism resulting in interstitial cystitis/bladder pain syndrome has not been fully elucidated, sensitization of bladder afferent pathways and a subsequent increase in sensory processing in the spinal cord have been proposed as important mechanisms inducing interstitial cystitis/bladder pain syndrome. Yoshimura et al. (2014), Int J Urol. 21 (0 1): 18-25. Therefore, pharmacological targets that could potentially suppress bladder afferent activity have been studied for the treatment of interstitial cystitis/bladder pain syndrome. Such treatments include opioids, adenosine receptor agonists, and GlyT inhibitors.

The present disclosure provides certain ORL-1 receptor modulators useful for treating interstitial cystitis/bladder pain syndrome.

### SUMMARY

The disclosure provides a method of treating interstitial cystitis/bladder pain syndrome in a human subject identified in need of such treatment, administering to the subject a therapeutically effective amount of a compound having the formula (I):

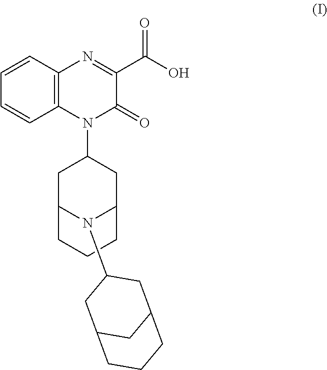 [SMILES: O=C(O)c1nc2ccccc2n(C2CC3CCCC(C2)N3C2CC3CCCC(C3)C2)c1=O]

or a pharmaceutically acceptable salt thereof. The compound of formula (I) includes all its stereoisomers (e.g., enantiomers) and polymorphic forms thereof.

In some embodiments, the disclosure provides a method of treating interstitial cystitis/bladder pain syndrome in a human subject identified in need of such treatment, administering to the subject a therapeutically effective amount of a compound having the formula (I′):

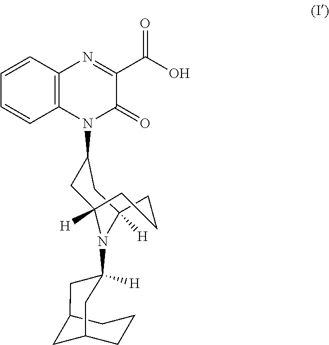

or a pharmaceutically acceptable salt thereof. As well appreciated by an ordinarily skilled person in the art, the compound of formula (I′) is a stereoisomer of the compound of formula (I).

In certain embodiments, the compound of formula (I) or formula (I′) is administered as a tosylate salt. For instance, in certain embodiments, the disclosure provides method of treating interstitial cystitis/bladder pain syndrome in a human subject in need of such treatment, comprising administering to the subject a therapeutically effective amount of a compound having the formula (IA):

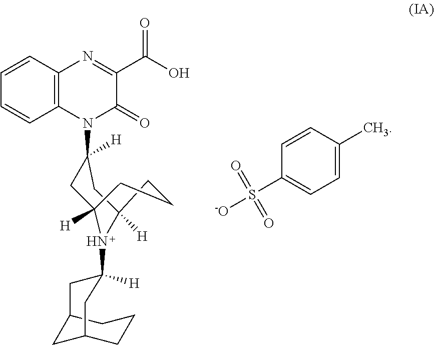

In one aspect, the disclosure provides a method of treating or relieving a symptom associated with interstitial cystitis/bladder pain syndrome in a human subject in need of such treatment, comprising administering to the subject a therapeutically effective amount of a compound of the formula (I) or (I′), or a pharmaceutically acceptable salt thereof. In one such embodiment, the symptom is visceral pain. In another such embodiment, the symptom is urinary urgency. In another such embodiment, administration of compound of the formula (I) or (I′), or a pharmaceutically acceptable salt thereof, results in reduction of occurrences of nocturia.

In particular embodiments the compound of formula (I) or (I′), or pharmaceutically acceptable salt thereof, is administered orally. Oral administration of the compound of formula (I) or pharmaceutically acceptable salt thereof results in high concentrations of the compound in the bladder.

In a certain embodiment, the present disclosure provides methods that are capable of simultaneously treating patients that suffer with sleep disorders and interstitial cystitis/bladder pain syndrome. Therefore, in one aspect, the disclosure provides methods of treating interstitial cystitis/bladder pain syndrome in a human subject who suffers from a sleep disorder, comprising administering to the subject a therapeutically effective amount of a compound having the formula (I) or (I′), or a pharmaceutically acceptable salt thereof. In some embodiments, the human subject suffers from insomnia. In some embodiments, the compound having the formula (I) or (I′), or a pharmaceutically acceptable salt thereof, is administered at night.

### BRIEF DESCRIPTION OF FIGURES

FIG. 1 shows that basal responses for the vehicle (saline), vehicle (CYP), ibuprofen/CYP and compound of formula (IA) groups were similar. FIG.1A shows nociceptive thresholds. FIG. 1B shows nociceptive scores. AUCs 1-6. FIG. 1C shows AUCs 1-6 g. FIG. 1D shows AUCs 6-26 g. Results are expressed as ±s.e.m. ^{NS} p>0.05, Kruskal-Wallis test, one-way ANOVA or two-way RM ANOVA with Sidak's post-test.

FIG. 2 shows time course of nociceptive threshold (g) after CYP or saline injection within both Vehicle groups. Results are expressed as±s.e.m.****p>0.0001, two-way RM ANOVA with Sidak's post-test.

FIG. 3 shows nociceptive scores (%) after 2 h (A), 3 h (B), 4 h (C) and 24 h (D) after CYP injection within both Vehicle groups. Results are expressed as ±s.e.m.**** p<0.0001, two-way RM ANOVA.

FIG. 4 shows the time course of AUC 1-6 g (A) and 6-26 g (B) after CYP or saline injection within both Vehicle groups. Results are expressed as ±s.e.m. *p<0.05 and**** p<0.0001, two-way RM ANOVA with Sidak's post-test.

FIG. 5 shows the time course of nociceptive threshold (g) after CYP injection in Vehicle and ibuprofen groups. Results are expressed as ±s.e.m.****p<0.0001, two-way RM ANOVA with Sidak's post-test.

FIG. 6 shows nociceptive scores (%), after 2 h (A), 3 h (B), 4 h (C) and 24 h (D) after CYP injection within Vehicle and ibuprofen groups. Results are expressed as s.e.m.****p<0.0001, two-way RM ANOVA.

FIG. 7 shows the time course of AUC 1-6 g (A) and 6-26 g (B) after CYP or saline injection within both Vehicle and ibuprofen groups. Results are expressed as ±s.e.m. *p<0.05 and****p<0.0001, two-way RM ANOVA with Sidak's post-test.

FIG. 8 shows the time course of nociceptive threshold (g) after CYP injection in Vehicle and the compound of formula (IA) groups. Results are expressed as ±s.e.m. ^{NS} p>0.05, **p<0.01, two-way RM ANOVA with Sidak's post-test.

FIG. 9 shows nociceptive scores (%), after 2 h (A), 3 h (B), 4 h (C) and 24 h (D) after CYP injection within Vehicle and the compound of formula (IA) groups. Results are expressed as ±s.e.m.**p<0.01,***p<0.001****and p<0.0001, two-way RM ANOVA.

FIG. 10 shows the time course of AUC 1-6 g (A) and 6-26 g (B) after CYP or saline injection within both Vehicle and the compound of formula (IA) groups. Results are expressed as s.e.m. ^{NS} p>0.05, *p<0.05, **p<0.01, **p<0.001, and ****p<0.0001, two-way RM ANOVA with Sidak's post-test.

### DETAILED DESCRIPTION

The disclosure provides a method of treating interstitial cystitis/bladder pain syndrome in a human subject identified as in need of such treatment, administering to the subject through administration of an agonist of the nociceptin opioid peptide receptor, also referred to as the ORL-1 receptor.

Identification of the ORL-1 receptor as distinct from the three long-known major classes of opioid receptors in the central nervous system-mu, kappa, and delta-resulted from experimentation on these opioid receptor classes. The ORL-1 receptor was identified and classified as an opioid receptor based only on amino acid sequence homology, as the ORL-1 receptor did not exhibit overlapping pharmacology with the classic mu opioid receptor. It was initially demonstrated that non-selective ligands having a high affinity for mu, kappa, and delta opioid receptors had low affinity for the ORL-1 receptor. This characteristic, along with the fact that an endogenous ligand had not yet been discovered, led to the term “orphan receptor.” See, e.g., Henderson et al., “The orphan opioid receptor and its endogenous ligand-nociceptin/orphanin FQ,” Trends Pharmacol. Sci. 188):293-300 (1997). Subsequent research led to the isolation and structure of the endogenous ligand of the ORL-1 receptor (i.e., nociceptin; also known as orphanin FQ or OFQ), a seventeen amino acid peptide structurally similar to members of the opioid peptide family. For a general discussion of ORL-1 receptors, see Calo′ et al., “Pharmacology of nociceptin and its receptor: a novel therapeutic target,” Br. J. Pharmacol. 129:1261-1283 (2000).

Through its interaction with ORL-1, nociceptin produces inhibitory activity of the micturition reflex in various animal models. Direct administration of nociceptin and peptide analogs of nociceptin to the bladder have been studied as a means of alleviating urinary incontinence, presumably through diminishing afferent signaling. However, there remains a need for effective treatment of lower urinary tract disorders such as interstitial cystitis/bladder pain based on non-peptidic small-molecule modulators of the ORL-1 receptor capable of being orally administered.

The inventors have discovered unexpectedly that interstitial cystitis/bladder pain can be ameliorated through the administration of a therapeutically effective amount of a compound of formula (I):

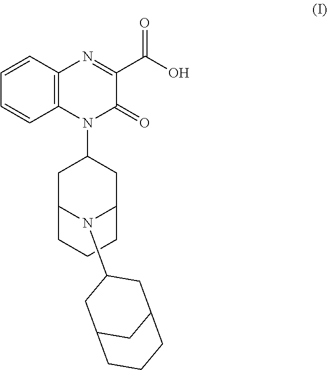 [SMILES: O=C(O)c1nc2ccccc2n(C2CC3CCCC(C2)N3C2CC3CCCC(C3)C2)c1=O]

or a pharmaceutically acceptable salt thereof.

Certain embodiments provide the compound of formula (I) or a pharmaceutically acceptable salt thereof as a single enantiomer (i.e., the compound of formula (I′)) having the structure depicted below:

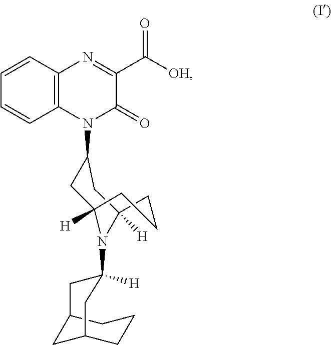

or a pharmaceutically acceptable salt thereof. In one embodiment, the pharmaceutically acceptable salt of the compound of formula (I′) is a para-toluenesulfonic acid salt (i.e., tosylate salt) of the compound of formula (I′), referred to as the compound of formula (IA), the structure of which is provided as follows:

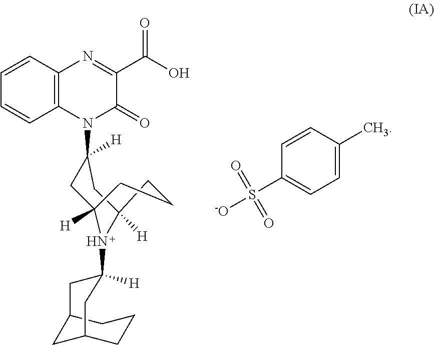

The compound of formulae (I), (I′), and (IA) are prepared as described in U.S. Pat. No. 8,476,221, which is hereby incorporated by reference. As referred to hereinafter, the compounds of the disclosure include the compounds of formulae (I) and (I′), or stereoisomers, pharmaceutically acceptable salts (e.g., the compound of formula (IA)), polymorphic forms, solvates, or hydrates thereof.

In certain embodiments, administration of the compound of formula (I) or (I′), or a pharmaceutically acceptable salt thereof also improves symptoms associated with interstitial cystitis/bladder pain, including, but not limited to, visceral pain and urinary urgency.

The terms “treatment of”, “treating”, and related terms as used herein include the amelioration, reduction, slowing, or cessation of a Condition or a symptom thereof by administration of an effective amount of a compound of formula (I) or (I′), or a pharmaceutically acceptable salt thereof. In some embodiments, treating includes inhibiting, for example, decreasing 1) the overall frequency of episodes of a Condition (e.g., interstitial cystitis/bladder pain); and/or 2) a symptom (e.g., visceral pain) thereof or reducing the severity of a Condition or a symptom thereof; and/or 3) the duration of the Condition or a symptom thereof.

In December 2019, the Food and Drug Administration (FDA) in the United States provided Guidance for Industry regarding treatment of interstitial cystitis/bladder pain syndrome According to the FDA guidance, the diagnosis of interstitial cystitis/bladder pain syndrome requires chronic bladder pain or discomfort; accompanying lower urinary tract symptoms(s), such as urinary frequency, urgency or nocturia; and exclusion of other disorders such as malignancy, endometriosis, chronic prostatitis and bladder outlet obstruction. The FDA guidance further indicates that some patients suffering from interstitial cystitis/bladder pain syndrome can experience constant bladder pain/discomfort. Other patients experience interstitial cystitis/bladder pain syndrome when voiding or as a burning sensation between voids as the bladder fills with urine.

The terms “prevention of”, “preventing”, and related terms as used herein include the avoidance of the onset of a Condition or a symptom thereof, or a decrease in incidence or frequency of a Condition or a symptom thereof, by administration of an effective amount of a compound of formula (I) or (I′), or a pharmaceutically acceptable salt thereof.

The term “effective amount”, when used in connection with methods for treating or preventing interstitial cystitis/bladder pain by administering a compound of formula (I) or (I′), or a pharmaceutically acceptable salt thereof (e.g., the compound of formula (IA)), refers to an amount of the compound administered to an animal that provides a therapeutic effect.

The compound of formula (I) or (I′), or a pharmaceutically acceptable salt thereof, exerts its beneficial effects through modulation of the ORL-1 receptor expressed on afferent nerve/fibers endings in the lower urinary tract. The terms “modulate”, “modulating”, and related terms as used herein with respect to the ORL-1 receptor mean the mediation of a pharmacodynamic response (e.g., interstitial cystitis/bladder pain) in an animal from (i) inhibiting or activating the receptor, or (ii) directly or indirectly affecting the normal regulation of the receptor activity. Compounds that modulate the receptor activity include agonists, partial agonists, biased agonists, antagonists, mixed agonists/antagonists, mixed partial agonists/antagonists and compounds which directly or indirectly affect regulation of the receptor activity. The compound of formula (I) or (I′), and pharmaceutically acceptable salts thereof, are partial agonists. As used herein, a compound that binds to a receptor and is only partly effective as an agonist is defined as a “partial agonist”. The partial agonists of the disclosure can achieve the desired therapeutic effects (e.g., treatment of interstitial cystitis/bladder pain), without concurrent side effects often associated with the administration of fill agonists.

The compound of formula (I) or (I′), or a pharmaceutically acceptable salt thereof, can be administered as a component of a composition that comprises a pharmaceutically acceptable carrier or excipient. Routes of administration include, but are not limited to, oral, intravesical, intradermal, intramuscular, intraperitoneal, parenteral, intravenous, subcutaneous, intranasal, epidural, transmucosal, buccal, gingival, sublingual, intraocular, intracerebral, intravaginal, transdermal (e.g., via a patch), rectal, by inhalation, or topical. In another embodiment, routes of administration include, but are not limited to, intravenous, intravesical, oral, or by inhalation. In another embodiment, the route of administration is oral. In another embodiment, the route of administration is intravesical. In another embodiment, the route of administration is intravenous. In another embodiment, the route of administration is by inhalation. In yet another embodiment, a compound of formula (I) or (I′), or a pharmaceutically acceptable salt thereof, is delivered in a controlled-release system or sustained-release system. Controlled- or sustained-release pharmaceutical compositions can have a common goal of improving drug therapy over that achieved by their non-controlled or non-sustained-release counterparts. For example, compared to their non-controlled or non-sustained-release counterparts, controlled- or sustained-release pharmaceutical compositions may achieve one or more of the following benefits, such as (but not limited to): reduced dosing frequency; increased duration of effects; increased magnitude of effects, e.g., by lowering Cₘₐₓ, or by increasing C_{average} at the site(s) of action; improved safety/tolerability; and improved patient compliance (less frequent dosing, better tolerability).

In one embodiment, a controlled- or sustained-release composition comprises a pharmaceutically acceptable amount of a compound of formula (I) or (I′), or a pharmaceutically acceptable salt thereof, to treat or prevent interstitial cystitis or a symptom thereof for an extended amount of time. Advantages of controlled- or sustained-release compositions include extended activity of the drug, reduced dosage frequency, and increased compliance. Administration of a compound of formula (I) or (I′), or a pharmaceutically acceptable salt thereof, can be by controlled-release or sustained-release means or by delivery devices that are known to those in the art. Examples include, but are not limited to, those described in U.S. Pat. Nos. 3,845,770, 3,916,899, 3,536,809, 3,598,123, 4,008,719, 5,674,533, 5,059,595, 5,591,767, 5,120,548, 5,073,543, 5,639,476, 5,354,556, and 5,733,566, each of which is incorporated herein by reference. Numerous other controlled-release or sustained-release delivery devices that are known to those in the art (see, e.g., Goodson, “Dental Applications,” in Medical Applications of Controlled Release, Vol. 2, Applications and Evaluation, Langer and Wise, eds., CRC Press, Chapter 6, pp. 115-138 (1984), hereafter “Goodson”). Other controlled- or sustained-release systems discussed in the review by Langer, Science 249:1527-1533 (1990) can be used. In one embodiment, a pump can be used (Langer, Science 249:1527-1533 (1990); Sefton, “Implantable Pumps,” in CRC Crit. Rev. Biomed. Eng. 14(3):201-240 (1987); Buchwald et al., “Long-term, Continuous Intravenous Heparin Administration by an Implantable Infusion Pump in Ambulatory Patients with Recurrent Venous Thrombosis,” Surgery 88:507-516 (1980); and Saudek et al., “A Preliminary Trial of the Programmable Implantable Medication System for Insulin Delivery,” New Engl. J. Med. 321:574-579 (1989)). In another embodiment, polymeric materials can be used (see Goodson; Smolen et al., “Drug Product Design and Performance,” Controlled Drug Bioavailability Vol. 1, John Wiley and Sons, New York (1984); Langer et al., “Chemical and Physical Structure of Polymers as Carriers for Controlled Release of Bioactive Agents: A Review,” J Macromol. Sci. Rev. Macromol. Chem. C23(1):61-126 (1983); Levy et al., “Inhibition of Calcification of Bioprosthetic Heart Valves by Local Controlled-Release Diphosphonate,” Science 228:190-192 (1985); During et al., “Controlled Release of Dopamine from a Polymeric Brain Implant: In Vivo Characterization,” Ann. Neurol. 25:351-356 (1989); and Howard et al., “Intracerebral drug delivery in rats with lesion-induced memory deficits,” J Neurosurg. 71:105-112 (1989)).

Suitable dosage forms can be used to provide controlled- or sustained-release of one or more active ingredients using, for example, hydropropyl methyl cellulose, ethyl cellulose, other polymer matrices, gels, permeable membranes, osmotic systems, multilayer coatings, microparticles, multiparticulates, liposomes, microspheres, or a combination thereof to provide the desired release profile in varying proportions. Suitable controlled- or sustained-release formulations known to those in the art, including those described herein, can be readily selected for use with the active ingredients of the disclosure. The disclosure thus encompasses single unit dosage forms suitable for oral administration such as, but not limited to, tablets, capsules, gelcaps, and caplets that are adapted for controlled- or sustained-release.

The compositions can optionally, but preferably, further comprise a suitable amount of a pharmaceutically acceptable excipient to provide the form for proper administration to the animal (e.g., a human). Such a pharmaceutical excipient can be a diluent, suspending agent, solubilizer, binder, disintegrant, preservative, coloring agent, lubricant, and the like. The pharmaceutical excipient can be a liquid, such as water or an oil, including those of petroleum, animal, vegetable, or synthetic origin, such as peanut oil, soybean oil, mineral oil, sesame oil, and the like. The pharmaceutical excipient can be saline, gum acacia, gelatin, starch paste, talc, keratin, colloidal silica, urea, and the like. In addition, auxiliary, stabilizing, thickening, lubricating, and coloring agents can be used. In one embodiment, the pharmaceutically acceptable excipient is sterile when administered to an animal. Water is a particularly useful excipient when a compound of formula (I) or (I′), or a pharmaceutically acceptable salt thereof, is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The compositions, if desired, can also contain minor amounts of wetting or emulsifying agents, or pH buffering agents. Specific examples of pharmaceutically acceptable carriers and excipients that can be used to formulate oral dosage forms are described in the Handbook of Pharmaceutical Excipient s, (Amer. Pharmaceutical Ass'n, Washington, DC, 1986), incorporated herein by reference. Other examples of suitable pharmaceutical excipients are described by Radebough et al., “Preformulation,” pp. 1447-1676 in Remington's Pharmaceutical Sciences Vol. 2 (Gennaro, ed., 19^{th} Ed., Mack Publishing, Easton, P A, 1995), incorporated herein by reference.

In one embodiment, the compound of formula (I) or (I′), or a pharmaceutically acceptable salt thereof, is formulated in accordance with routine procedures as a composition adapted for oral administration to human beings. A compound of formula (I) or (I′), or a pharmaceutically acceptable salt thereof, to be orally delivered can be in the form of tablets, capsules, gelcaps, caplets, lozenges, aqueous or oily solutions, suspensions, granules, microparticles, multiparticulates, powders, emulsions, syrups, or elixirs, for example. When a compound of formula (I) or (I′), or a pharmaceutically acceptable salt thereof, is incorporated into oral tablets, such tablets can be compressed, tablet triturates, enteric-coated, sugar-coated, film-coated, multiply compressed, or multiply layered. Techniques and compositions for making solid oral dosage forms are described in Pharmaceutical Dosage Forms: Tablets (Lieberman et al., eds., 2^{nd} Ed., Marcel Dekker, Inc., 1989 and 1990). Techniques and compositions for making tablets (compressed and molded), capsules (hard and soft gelatin) and pills are also described by King, “Tablets, Capsules, and Pills,” pp. 1553-1593 in Remington's Pharmaceutical Sciences (Osol, ed., 16^{th} Ed., Mack Publishing, Easton, P A, 1980).

Liquid oral dosage forms include aqueous and non-aqueous solutions, emulsions, suspensions, and solutions and/or suspensions reconstituted from non-effervescent granules, optionally containing one or more suitable solvents, preservatives, emulsifying agents, suspending agents, diluents, sweeteners, coloring agents, flavoring agents, and the like. Techniques and composition for making liquid oral dosage forms are described in Pharmaceutical Dosage Forms: Disperse Systems (Lieberman et al., eds., 2^{nd} Ed., Marcel Dekker, Inc., 1996 and 1998).

An orally administered compound of formula (I) or (I′), or a pharmaceutically acceptable salt thereof, can contain one or more excipients, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

The compositions can take the form of solutions, suspensions, emulsions, tablets such as an orally disintegrating tablet (ODT) or a sublingual tablet, pills, pellets, capsules, capsules containing liquids, powders, sustained-release formulations, suppositories, emulsions, aerosols, sprays, suspensions, microparticles, multiparticulates, rapidly dissolving films or other forms for oral or mucosal administration, or any other form suitable for use. In one embodiment, the composition is in the form of an ODT (see, e.g., U.S. Pat. Nos. 7,749,533 and 9,241,910). In another embodiment, the composition is in the form of a sublingual tablet (see, e.g., U.S. Pat. Nos. 6,572,891 and 9,308,175). In another embodiment, the composition is in the form of a capsule (see, e.g., U.S. Pat. No. 5,698,155). In another embodiment, the composition is in a form suitable for buccal administration, e.g., as a tablet, lozenge, gel, patch, or film, formulated in a conventional manner (see, e.g., Pather et al., “Current status and the future of buccal drug delivery systems,” Expert Opin. Drug Deliv. 5(5):531-542 (2008)). In another embodiment, the composition is in a form suitable for gingival administration, e.g., as a polymeric film comprising polyvinyl alcohol, chitosan, polycarbophil, hydroxypropylcellulose, or Eudragit S-100, as disclosed by Padula et al., “In Vitro Evaluation of Mucoadhesive Films for Gingival Administration of Lidocaine,” AAPS PharmSciTech 14(4):1279-1283 (2013). In another embodiment, the composition is in a form suitable for intraocular administration.

In one embodiment, the compounds of formula (I) or (I′), or a pharmaceutically acceptable salt thereof, is formulated for parenteral administration. When a compound of formula (I) or (I′), or a pharmaceutically acceptable salt thereof, is to be injected parenterally, it can be, e.g., in the form of an isotonic sterile solution. In one embodiment, the parenteral administration comprises the compound of formula (IA).

When a compound of formula (I) or (I′), or a pharmaceutically acceptable salt thereof (such as, the compound of formula (IA)) is administered parenterally, the formulation for parenteral administration can be in the form of a suspension, solution, emulsion in an oily or aqueous vehicle. Such formulations can further comprise pharmaceutically necessary additives such as one or more stabilizing agents, suspending agents, dispersing agents, buffers, and the like. A compound of formula (I) or (I′), or a pharmaceutically acceptable thereof (such as, the compound of formula (IA)), can also be in the form of a powder for reconstitution as an injectable formulation.

In another embodiment, the compounds of formula (I) or (I′), or a pharmaceutically acceptable thereof (e.g., the compound of formula (IA)), can be formulated for intravenous administration. In certain embodiments, compositions for intravenous administration comprise sterile isotonic aqueous buffer. Where necessary, the compositions can also include a solubilizing agent. A compound of formula (I) or (I′), or a pharmaceutically acceptable salt thereof (e.g., the compound of formula (IA)), for intravenous administration can optionally include a local anesthetic such as benzocaine or prilocaine to lessen pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where a compound of formula (I) or (I′), or a pharmaceutically acceptable salt thereof (e.g., the compound of formula (IA)), is to be administered by infusion, it can be dispensed, for example, with an infusion bottle containing sterile pharmaceutical grade water or saline. Where a compound of formula (I) or (I′), or a pharmaceutically acceptable salt thereof (e.g., the compound of formula (IA)), is administered by injection, an ampule of sterile water for injection or saline can be provided so that the ingredients can be mixed prior to administration.

The compound of formula (I) or (I′) is primarily excreted from the urine largely unchanged. See Example 2. Therefore, the compound of formula (I) or (I′) shows high concentrations in the bladder following administration. For instance, when administered orally, the compound of formula (I) or (I′) excreted in the urine ranges from about 30% to about 95%, depending on the dose administered. Additionally, the concentration of the compound of formula (I) or (I′) in the urine is sustained including up to 24 hours following oral administration at certain doses. In particular embodiments, the concentration of the compound of formula (I) or (I′) 12 hours following oral administrations is greater than 100 nM. In other embodiments, the concentration of the compound of formula (I) or (I′) 12 hours following oral administrations is greater than 500 nM. In other embodiments, the concentration of the compound of formula (I) or (I′) 12 hours following oral administrations is greater than 1,000 nM. In other embodiments, the concentration of the compound of formula (I) or (I′) 12 hours following oral administrations is greater than 5,000 nM. In other embodiments, the concentration of the compound of formula (I) or (I′) 12 hours following oral administrations is greater than 10,000 nM. In particular embodiments, the concentration of the compound of formula (I) or (I′) 12 hours after oral administration can range from about 100 nM to about 30,000 nM. In other embodiments, the concentration of the compound of formula (I) or (I′) 12 hours after oral administration can range from about 500 nM to about 15,000 nM. In other embodiments, the concentration of the compound of formula (I) or (I′) 12 hours after oral administration can range from about 1,000 nM to about 10,000 nM.

In some embodiments, the compound of formula (I) or (I′) is administered in the form of a pharmaceutically acceptable salt. The term “pharmaceutically acceptable salt”, as used herein, is any pharmaceutically acceptable salt that can be prepared from a compound of formula (I). Illustrative salts include, but are not limited, to sulfate, citrate, acetate, trifluoroacetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucoronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. In one embodiment, the pharmaceutically acceptable salt is a hydrochloride salt, a sulfate salt, a sodium salt, a potassium salt, a benzene sulfonic acid salt, a para-toluenesulfonic acid salt, or a fumaric acid salt. In another embodiment, the pharmaceutically acceptable salt is a hydrochloride salt or a sulfate salt. In another embodiment, the pharmaceutically acceptable salt is a hydrochloride salt. In another embodiment, the pharmaceutically acceptable salt is a sulfate salt. In another embodiment, the pharmaceutically acceptable salt is a sodium salt. In another embodiment, the pharmaceutically acceptable salt is a potassium salt. In another embodiment, the pharmaceutically acceptable salt is a fumaric acid salt. In another embodiment, the pharmaceutically acceptable salt is a para-toluenesulfonic acid salt (also known as, “tosylate salt”). In another embodiment, the pharmaceutically acceptable salt is a choline salt.

In another embodiment, the pharmaceutically acceptable para-toluenesulfonic acid salt contains one equivalent of a compound of formula (I) or (I′) and about 1.0 equivalent of toluenesulfonic acid, e.g., from about 0.8 to about 1.2 equivalents of para-toluenesulfonic acid in one embodiment, from about 0.9 to about 1.1 equivalents of para-toluenesulfonic acid in another embodiment, from about 0.93 to about 1.07 equivalents of para-toluenesulfonic acid in another embodiment, from about 0.95 to about 1.05 equivalents of para-toluenesulfonic acid in another embodiment, from about 0.98 to about 1.02 equivalents of para-toluenesulfonic acid in another embodiment, or from about 0.99 to about 1.01 equivalents of para-toluenesulfonic acid in another embodiment. In another embodiment, the pharmaceutically acceptable para-toluenesulfonic acid salt contains about one equivalent of a compound of formula (I) or (I′) and about one equivalent of para-toluenesulfonic acid, i.e., is a mono-tosylate salt. In another embodiment, the pharmaceutically acceptable para-toluenesulfonic acid salt contains one equivalent of a compound of formula (I) or (I′). In another embodiment, the pharmaceutically acceptable para-toluenesulfonic acid salt contains one equivalent of the compound of formula (I′), that is, the mono-tosylate salt of the compound of formula (I′), i.e., the compound of formula (IA), as follows:

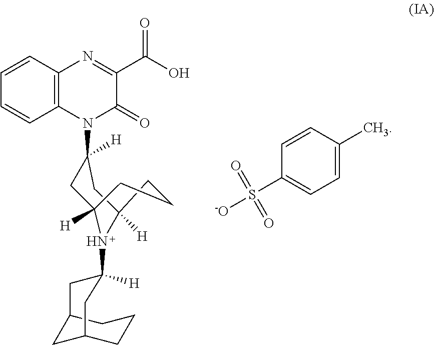

The methods of the disclosure provided herein also encompass the use of any solvate of the compounds of formula (I), (I′), or a pharmaceutically acceptable salt thereof. “Solvates” are generally known in the art, and are considered herein to be a combination, physical association and/or solvation of a compound of formula (I), (I′), or a pharmaceutically acceptable salt thereof. This physical association can involve varying degrees of ionic and covalent bonding, including hydrogen bonding. When the solvate is of the stoichiometric type, there is a fixed ratio of the solvent molecule to the compound of formula (I), (I′), or a pharmaceutically acceptable salt thereof. A compound of formula (I), (I′), or a pharmaceutically acceptable salt thereof can be present as a solvated form with a pharmaceutically acceptable solvent, such as water, methanol, ethanol, and the like.

The methods of the disclosure provided herein also encompass the use of any crystalline form (or polymorphic form) of the compounds of formula (I), (I′), or a pharmaceutically acceptable salt thereof. The term “crystalline” and related terms used herein, when used to describe a substance, component or product, means that the substance, component or product is substantially crystalline as determined by X-ray diffraction, microscopy, polarized microscopy, or other known analytical procedure known to those skilled in the art. The term “polymorph,” as used herein, refers to crystalline forms of a compound having different unit cell structures in crystals, originating from a variety of molecular conformations and molecular packing. Polymorphs of a single compound can have one or more different chemical, physical, mechanical, electrical, thermodynamic, and/or biological properties from each other. Differences in physical properties exhibited by polymorphs can affect pharmaceutical parameters such as storage stability, compressibility, density (important in composition and product manufacturing), dissolution rates (an important factor in determining bio-availability), solubility, melting point, chemical stability, physical stability, powder flowability, water sorption, compaction, and particle morphology. Differences in stability can result from changes in chemical reactivity (e.g., differential oxidation, such that a dosage form discolors more rapidly when comprised of one polymorph than when comprised of another polymorph), or mechanical changes (e.g., crystal changes on storage as a kinetically favored polymorph converts to a thermodynamically more stable polymorph) or both (e.g., one polymorph is more hygroscopic than the other.

In certain embodiments, the compound of formula (IA) has the crystalline form referred to as Form A, Form B, Form C, Form D, or Form E, as described in WO 2020/157691, the contents of which are incorporated by reference. In some embodiments, the compound of formula (IA) is of crystalline Form A. In other embodiments, the compound of formula (IA) is of crystalline Form B. In other embodiments, the compound of formula (IA) is of crystalline Form C. In other embodiments, the compound of formula (IA) is of crystalline Form D. In other embodiments, the compound of formula (IA) is of crystalline Form E.

The amount by weight of the administered “dose”, “dosage”, and related terms as used herein refers to the free acid and free base form of a compound of formula (I) or (I′), i.e., the non-salt form. For example, a 10.0 mg dose of the non-salt form of the compound of formula (I) or (I′) means that 10.0 mg is actually administered. However, by way of example, a 10.0 mg dose of, e.g., the monohydrochloride or the 1:1 by moles hydrochloric acid salt of the compound of formula (1) means that 10.84 mg of said compound is actually administered, which 10.84 mg provides 10.00 mg of the non-salt form of the compound of formula (I) or (I′) (0.0229 mmoles) and 0.84 mg of hydrochloric acid (0.0229 mmoles). Likewise, a 10.00 mg dose of, e.g., the mono-tosylate salt (1:1 by moles para-toluenesulfonic acid salt) of the compound of formula (IA), means that 13.93 mg of said compound is actually administered, which 13.93 mg provides 10.00 mg of the non-salt form of the compound of formula (I′) (0.0229 mmoles) and 3.93 mg of para-toluenesulfonic acid (0.0229 mmoles).

In terms of a method for treating or preventing a Condition or a symptom in a human subject, suitable effective dosage amounts of the compound of formula (I) or (I′), or a pharmaceutically acceptable salt or thereof, are from about 0.00002 mg/kg to about 10 mg/kg of body weight of the human subject per day in one embodiment, from about 0.00025 mg/kg/day to about 5 mg/kg/day in another embodiment, from about 1.5 mg/kg/day to about 3 mg/kg/day in another embodiment, from about 0.2 mg/kg/day to about 2 mg/kg/day in another embodiment, from about 2.5 mg/kg/day to about 10.0 mg/kg/day in another embodiment, and from about 3.0 mg/kg/day to about 10 mg/kg/day in another embodiment. In certain embodiments, suitable effective dosage amounts of the compound of formula (I) or (I′), or a pharmaceutically acceptable salt thereof (e.g., the compound of formula (IA)), are from about 0.00002 mg/kg/day to about 10 mg/kg/day, from about 0.001 mg/kg/day to about 10 mg/kg/day. In another embodiment, the effective dosage amount is about 1.0 mg/kg/day or less. It is to be understood that for these dosage amounts, the term “day” means a 24-hour cycle beginning at the time of administration of a compound of formula (I) or (I′), or a pharmaceutically acceptable salt thereof. It is generally understood that it is possible to extrapolate suitable doses between animals and humans (Nair et al., J. Basic Clin. Pharm., March-May 2016; 7(2):27-31).

In embodiments where the compound of formula (I) or (I′), or a pharmaceutically acceptable salt thereof (e.g., the compound of formula (IA)), is administered orally, a suitable effective dosage amount of the compound as a single dose is from about 0.001 mg to about 300 mg, from about 0.005 mg to about 250 mg, from about 0.01 mg to about 200 mg, from about 0.05 mg to about 150 mg, from about 0.075 mg to about 50 mg, from about 0.10 mg to about 10 mg. In one embodiment, the compound of the disclosure is administered as a single dose in a non-controlled or non-sustained release formulation. In another embodiment, the effective dosage amount of the compound of the disclosure is administered as multiple doses in non-controlled or non-sustained release formulations.

In certain embodiments, about 0.05 mg, about 0.06 mg, about 0.07 mg, about 0.08 mg, about 0.09 mg, about 0.100 mg, about 0.120 mg, about 0.125 mg, about 0.150 mg, about 0.175 mg, about 0.200 mg, about 0.225 mg, about 0.250 mg, about 0.275 mg, about 0.30 mg, about 0.35 mg, about 0.40 mg, about 0.45 mg, about 0.50 mg, about 0.55 mg, about 0.60 mg, about 0.65 mg, about 0.70 mg, about 0.75 mg, about 0.80 mg, about 0.85 mg, about 0.90 mg, about 0.95 mg, about 1.00 mg, about 1.25 mg, about 1.50 mg, about 1.75 mg, about 2.00 mg, about 2.25 mg, about 2.50 mg, about 2.75 mg, about 3.00 mg, about 3.25 mg, about 3.50 mg, about 3.75 mg, about 4.0 mg, about 4.5 mg, about 5.0 mg, about 5.5 mg, about 6.0 mg, about 6.5 mg, about 7.0 mg, about 7.5 mg, about 8.0 mg, about 9.0 mg, or about 10 mg of the compound of formula (I) or (I′), or a pharmaceutically acceptable salt thereof (e.g., the compound of formula (IA)), is administered orally to a human subject in need thereof. As known to those in the art, for a human animal, a single daily dose (in mg) can be converted to a mg/kg/day dosage amount by dividing the mg dose by 60 kg, the art-recognized average mass of a human animal. For example, a single daily human dose of 12 mg is so-converted to a dosage amount of about 0.20 mg/kg/day.

In certain embodiments, a controlled-release composition comprising a therapeutically effective amount of a compound of the disclosure is administered as a single dose or in multiple doses. The controlled-release composition may contain up to 100 times of the dosage amount of the compound of formula (I) or (I′), or a pharmaceutically acceptable salt thereof (e.g., the compound of formula (IA)), that is used for a non-controlled or non-sustained-release formulation.

In some embodiments, the compound of formula (I) or (I′), or a pharmaceutically acceptable salt thereof can be administered once daily. In some such embodiments, the compound of formula (I) is administered nightly. As shown in Example 2, daily administration of the compound of formula (I), (I′), or (IA) results in urine concentrations well in excess of the in vitro activity of the compounds.

In addition to its beneficial effects of treating conditions associated with interstitial cystitis, the compound of formula (I), (I′) or (IA) also, when administered at sufficient dose levels, is capable of inducing drowsiness and treating sleep disorders. See U.S. Publication No. 2020/0345726, which is hereby incorporated by reference. Patients suffering from symptoms associated with interstitial cystitis often suffer from poor sleep quality, insomnia and/or nocturia. In some embodiments, the patients suffering from sleep disorders are females 50 years of age or older. In other embodiments, the patients suffering from sleep disorders are males 50 years of age or older. Nightly administration of the compound of formula (I) or (I′), or a pharmaceutically acceptable salt thereof, improved both sleep quality and symptoms associated with interstitial cystitis. In particular embodiments, the compound of formula (I) or (I′), or a pharmaceutically acceptable salt thereof, is administered by the patient each night prior to sleep. For instance, the compound of formula (I) or (I′), or a pharmaceutically acceptable salt thereof, can be administered from about 1 minute to about 3 hours prior to sleep. In some embodiments, the compound of formula (I) or (I′), or a pharmaceutically acceptable salt thereof, can be administered from about 5 minutes to about 60 minutes prior to sleep. In other embodiments, the compound of formula (I) or (I′), or a pharmaceutically acceptable salt thereof, can be administered from about 10 minutes to about 30 minutes prior to sleep.

In one embodiment, an effective dose or dosage amount of the compound of formula (I) or (I′), or a pharmaceutically acceptable salt thereof, is administered about 60 minutes before a human's median habitual bedtime. In another embodiment, an effective dose or dosage amount is administered about 45 minutes before a human's median habitual bedtime. In another embodiment, an effective dose or dosage amount is administered about 30 minutes before a human's median habitual bedtime. In another embodiment, an effective dose or dosage amount is administered about 20 minutes before a human's median habitual bedtime. In another embodiment, an effective dose or dosage amount is administered about 20 minutes or less before a human's median habitual bedtime. In another embodiment, an effective dose or dosage amount is administered about 15 minutes before a human's median habitual bedtime. In another embodiment, an effective dose or dosage amount is administered about 15 minutes or less before a human's median habitual bedtime. In another embodiment, an effective dose or dosage amount is administered about 10 minutes before a human's median habitual bedtime. In another embodiment, an effective dose or dosage amount is administered about 10 minutes or less before a human's median habitual bedtime. In another embodiment, an effective dose or dosage amount is administered about 5 minutes before a human's median habitual bedtime.

In other embodiments, the compound of formula (I) or (I′), or a pharmaceutically acceptable salt thereof, can be administered multiple times during the day. For instance, the compound of formula (I), or a pharmaceutically acceptable salt thereof, can be administered twice daily or three times daily. In embodiments where the compound is administered multiple times daily, each dose may be administered in the same amount or in different amounts. In some embodiments, the dose of the compound of formula (I) or (I′), or pharmaceutically acceptable salt thereof, is administered at a higher dose than the other doses provided earlier in the day. In some embodiments, the compound of formula (I) or (I′), or pharmaceutically acceptable salt thereof, is administered twice daily, approximately every 12 hours. In other embodiments, the compound of formula (I) or (I′), or pharmaceutically acceptable salt thereof, is administered three times daily, approximately every 8 hours.

In particular embodiments, the compound of formula (I) or (I′), or a pharmaceutically acceptable salt thereof, is administered twice daily, wherein the second dose (i.e., the second therapeutically effective amount) is administered prior to bedtime, as set forth above. In some such embodiments, the second dose is administered at a higher amount than the first dose (i.e., the first therapeutically effective amount). For instance, the second dose can be administered at an amount of about 1.5-fold, 2-fold, 3-fold-, 5-fold, 10-fold, 20-fold, 50-fold. 100-fold or 1000-fold higher than the first dose. In some embodiments, the second dose can be administered in an amount from about 1.5-fold to about 10-fold higher than the first dose. In other embodiments, the second dose can be administered in an amount from about 1.5-fold to about 100-fold higher than the first dose. In other embodiments, the second dose can be administered in an amount from about 1.5-fold to about 1000-fold higher than the first dose. In other embodiments, the second dose can be administered in an amount from about 3-fold to about 100-fold higher than the first dose. In other embodiments, the second dose can be administered in an amount from about 3-fold to about 1000-fold higher than the first dose. In other embodiments, the second dose can be administered in an amount from about 5-fold to about 100-fold higher than the first dose. In other embodiments, the second dose can be administered in an amount from about 5-fold to about 1000-fold higher than the first dose. Such dosing schedules ensure that the first dose is effective at treating interstitial cystitis and symptoms associated with interstitial cystitis without causing residual drowsiness, whereas the second dose is effective at treating interstitial cystitis and symptoms associated with interstitial cystitis and causing drowsiness or inducing sleep in a human subject. In certain embodiments, both the first dose and the second dose are administered through (same or different) non-controlled or non-sustained release formulations. In other embodiments, the first dose is administered through a controlled or non-sustained release formulation, and the second dose is administered through a non-controlled or non-sustained release formulation.

In another embodiment, a composition comprising a compound of the disclosure is useful as a medicament in the treatment of human subjects suffering from both interstitial cystitis/bladder pain and a particular sleep disorder. Such sleep disorders include, but are not limited to, an insomnia condition, a hypersomnia condition, a circadian rhythm sleep-wake disorder, an alcohol-induced sleep disorder, or any combination thereof. Other sleep disorders include an alcohol-induced sleep disorder (e.g., insomnia-type alcohol-induced sleep disorder, daytime sleepiness type alcohol-induced sleep disorder, parasomnia type alcohol-induced sleep disorder, and mixed type alcohol-induced sleep disorder); insomnia in alcohol use disorder; sleep disturbances associated with alcohol cessation (e.g., insomnia associated with alcohol cessation); or any combination thereof.

The methods for treating or preventing interstitial cystitis/bladder pain in a patient in need thereof can further comprise co-administering to the patient a compound of formula (I) or (I′), or a pharmaceutically acceptable salt thereof, a second therapeutic agent. In one embodiment, the second therapeutic agent is also administered in an effective amount for achieving its desired therapeutic effects. In one embodiment, the second therapeutic agent is an antimuscarinic agent. In some such embodiments, the antimuscarinic agent is oxybutynin. In other such embodiments, the antimuscarinic agent is tolterodine. In other such embodiments, the antimuscarinic agent is trospium. In other such embodiments, the antimuscarinic agent is solifenacin. In other such embodiments, the antimuscarinic agent is darifenacin. In other such embodiments, the antimuscarinic agent is flavoxate.

### Examples

### Example 1: Cyclophosphamide-Induced Visceral Pain Model for Interstitial Cystitis/Bladder Pain Syndrome

Cyclophosphamide (CYP)-induced inflammatory visceral pain is a well-established experimental model for interstitial cystitis/bladder pain syndrome. Intraperitoneal (i.p.) injection with CYP could reproduce most features of interstitial cystitis/bladder pain syndrome in humans such as increasing voiding frequency, decreasing urine volume per void and visceral pain (Takagi-Matsumoto et al., 2004; Boucher et al., 2000; Smaldone et al., 2009). Consequently, the CYP-induced cystitis model is widely used in rats for evaluation of candidate compounds (Juszczak et al., 2007). CYP is an antineoplastic agent that is widely used for the treatment of malignancies such as lymphoma and leukemia. CYP administered in the peritoneum is converted to the toxic metabolite acrolein in the kidney, which accumulates in the bladder and injures the urinary tract (Cox, 1979).

In female Sprague-Dawley rats, we showed that a single i.p. injection of CYP induces urinary bladder inflammation and behaviors associated with visceral pain, which peak shortly after CYP injection and persist for up to 24 hours (Lluel et al., 2010 and Augé et al., 2011 and 2013). The CYP-induced visceral pain is characterized by both referred mechanical hyperalgesia (increased sensitivity to pain) and allodynia (nociceptive response elicited by a normally innocuous stimulus) (Augé et al., 2013).

The aim of the present study was to evaluate the effects of single oral administration of the compound of formula (IA) at 30 mg/kg on visceral pain in the acute CYP-induced cystitis model in female Sprague-Dawley rats. Ibuprofen (300 mg/kg, p.o.) was used as positive substance. Visceral pain was assessed blinded by von Frey testing.

### 1.1 Study Design

### 1.1.1 Protocol Design

At Day 1 (“D1”), the day prior induction of cystitis, rats were acclimatized to the individual Plexiglas box for a minimum of 30 min and to the application of the von Frey filaments in order to decrease the level of stress due to the new environment. Acclimatization was performed exactly under the same experimental condition as for pain evaluation (see § 2.4.1 for details).
At Day 0 (“D0”), von Frey testing was first performed in order to obtain basal values for nociceptive behavior.
At D0, just prior cystitis induction, pharmacological treatment was performed as described in § 2.4.2.
At D0, acute cystitis was induced by CYP injection. Control rats received saline (for details see § 2.4.3) (DO, T=0).

At D0 and D1, von Frey testing was performed 2, 3, 4 and 24 hours after cystitis induction in order to analyze test and positive substances effects on CYP-induced visceral pain (D0, T=+2 h, T=+3 h, T=+4 h and D1, T=+24 h). Just after the last von Frey testing, rats were euthanized.

### 1.1.2 Experimental Groups

Four (4) experimental groups were included as described in the table below:

TABLE 1

| Group | Treatment (p.o.) | Injection (i.p.) | n |
|---|---|---|---|
| 1 | Vehicle | Saline | 10 |
| 2 | Vehicle | CYP | 10 |
| 3 | Compound of formula (IA) (30 mg/kg) | CYP | 10 |
| 4 | Ibuprofen (300 mg/kg) | CYP | 10 |

### 1.2 Animals

All experiments were conducted in accordance with the European Community Council Directive 2010/63/UE and the French Ministry for Agriculture, Agrifood and Forestry Decree 2013-118. Experimental protocols were reviewed by CEEA-122 Ethical Committee for Protection of Animals used for Scientific Purposes and approved by French Ministry for National Education, Higher Education and Research under the number APAFIS #21140-2019062010159601 v2

Female Sprague-Dawley rats were acclimated to the laboratory conditions for at least 3 days before the start of experiments. Animals were housed in groups of 3 in polysulfone type Sealsafe plus 1291H cages (Tecniplast, Lyon, France) on a bed of wood chips (Souralit, Girona, Spain) with free access to food (Rodent Maintenance Diet A04/10 from Safe) and water (0.2 μm filtered water). Species appropriate environmental enrichment (Aspen brick, Plexx, Uden, Netherlands) was added in the cages. The animal house was maintained under artificial lighting (12 h) between 7:00 am to 7:00 pm in a controlled ambient temperature of 22±2° C., and relative humidity maintained at 55±10%.

### 1.3 Substances

### 1.3.1 Test Substance and Vehicle

### 1.3.1.1 Preparation of Formulation

A formulation of the compound of formula (IA) was prepared at a final concentration of 6 mg/mL (based on free base form) at room temperature. Appropriate mass of the compound of formula (IA) was weighed and vehicle was added slowly in a porcelain mortar. Powder was ground with a pestle until a suspension was obtained. Aliquots of suspension were made (1 aliquot/administration) and kept at +4° C. for a maximum of 3 days. On each experimental day, the suspension was allowed to equilibrate to room temperature for at least 30 min prior to administration.

### 1.3.1.2 Vehicle

Vehicle was 0.5% methylcellulose (MC) with a viscosity of 400 cP. It was prepared in water for injection (WFI) and kept at 4° C. for 1 week. MC (batch n° SLBR8963V) was purchased from Sigma-Aldrich (Saint-Quentin Fallavier, France).

### 1.3.2 Positive Substance

Ibuprofen was prepared fresh on the day of each administration at a final concentration of 60 mg/mL (free base form). Appropriate mass of Ibuprofen was weighed and dissolved in vehicle at room temperature. The solution was vortexed for 15-30 sec, then sonicated for 1 min and then vortex for an additional 15-30 sec. Ibuprofen was purchased from Sigma-Aldrich (batch n ° BCBR4459V).

### 1.3.3 Anesthetic/Euthanasia Substance

Dolethal® was purchased from Vetoquinol via Centravet (Lapalisse, France, batch n° 9C2800C).

1.3.4 Additional substances

CYP was purchased from Sigma-Aldrich (batch n° MKCG5464). Saline was purchased from B-Braun via Centravet (batch n° 18465450). WFI was supplied by Cooper (Melun, France; batch n° 19MD16GA).

### 1.4 Study Protocols

### 1.4.1 Assessment of Visceral Pain Using Von Frey Filaments

Standardized conditions including single-experimenter testing of all animals were applied to minimize variability behavior-based pain testing.

Visceral pain was evaluated in a blinded manner by applying to the lower abdomen, close to the urinary bladder, a set of 8 calibrated von Frey filaments of increasing forces (1, 2, 4, 6, 8, 10, 15 and 26 g) with an interstimulus interval of 5 seconds. Prior to testing, the abdominal area designed for mechanical stimulation of each animal was shaved. Animals were placed on a raised wire mesh floor under individual transparent Plexiglas box and acclimatized for at least 30 minutes before starting the von Frey test. Filaments were then applied for 1-2 seconds through the mesh floor with enough strength to cause the filament to slightly bend. Each filament was tested 3 times. Care was taken to stimulate different areas within the lower abdominal region in the vicinity of the urinary bladder to avoid desensitization.

Nociceptive behaviors were scored for each animal and each filament as follows:

TABLE 2

| Score | Behavior |
|---|---|
| 0 | no response |
| 1 | retraction of the abdomen |
| 2 | trampling or change of position |
| 3 | flinching or abdominal curvature or licking of the |
| | site stimulated with von Frey filaments |

### 1.4.2 Pharmacological Treatments

Prior to the experiment, the animals were randomly assigned to treatment groups using the block method, which consists of distributing at least one animal per treatment or control in the same block.

The compound of formula (IA) (30 mg/kg), ibuprofen (300 mg/kg) and vehicle were administered orally (p.o.) once, just prior CYP or saline injection. On the morning of each experimental day, rats were weighed and administrations were performed at a volume of 5 mL/kg.

### 1.4.3 Induction of Acute Cystitis

To induce acute cystitis, a single i.p. injection of CYP at a dose of 150 mg/kg in a final volume of 5 mL/kg (in saline) was performed. Control rats received saline under the same experimental conditions than CYP.

### 1.5 Laboratory Equipment

Mechanical stimulation was performed using von Frey filaments of different forces (reference: Bio-VF-M; BioSeb ID Tech, Vitrolles, France). Filaments were purchased pre-calibrated. Animals were weighed using a LS620C balance (PRECISA, Dietikon, Switzerland).

### 1.6 Expression and Analysis of Results

All raw data were compared with raw data by two persons before data analysis. Results are expressed as mean values±standard error of the mean (s.e.m.).

### 1.6.1 Visceral Pain

TABLE 3

| Nociceptive parameter | Expression | Description |
|---|---|---|
| Nociceptive threshold | g | von Frey force for which the stimulus is |
| | | perceived as painful (score ≥1 is obtained) |
| Nociceptive scores | % | % of the maximal response for each filament |
| | | (on the 3 pooled applications*) |
| Area under the curve (AUC) 1-6 g | % scores × | calculated from the curve of % nociceptive |
| - allodynia - | g | scores plotted against painless (1 to 6 g) von |
| | | Frey forces |
| Area under the curve (AUC) 6-26 g | | calculated from the curve of % nociceptive |
| - hyperalgesia - | | scores plotted against painful (6 to 26 g) von |
| | | Frey forces |

*For example, at a given von Frey force, for an animal with a score of 1 at the first application, 1 at the second and 2 at the third, the summation of its scores is 4. The maximal pooled score being 9 (3 + 3 + 3), a pooled score of 4 equals 44% of the maximal response (100 × 4/9).

### 1.7 Statistical Analysis

Statistical analysis and graphs were performed using GraphPad Prism© (GraphPad Software Inc., La Jolla, CA, USA). A p value <0.05 was accepted for statistical significance.

Except when two-way analysis was applied, before carrying out any statistical test, the data were tested for normal distribution (Shapiro-Wilk normality test) and their variance evaluated (F test or Bartlett's test for two or more groups, respectively). The appropriate statistical test was consequently applied.

### 1.7.1 Comparison of Basal Nociceptive Responses

To verify that all groups had similar basal values, grouped comparison followed by post hoc test between all groups was made. Post hoc test was run to analyze where potential difference occurred between groups. For clarity of the graphs, post hoc results were not indicated when p>0.05.
Kruskal-Wallis test was used as non-parametric analysis of variance (ANOVA) with Dunn's multiple comparisons.
One-way ANOVA test was used as parametric ANOVA with Holm-Sidak's multiple comparisons.
Two-way ANOVA with repeated measures (RM) followed by Holm Sidak's multiple comparisons was used for nociceptive scores.

### 1.7.2 Time Course Analysis of Effects of CYP

To analyze CYP effects, individual pairwise comparison was made between Vehicle/Saline and Vehicle/CYP groups.

Two-way RM ANOVA with Sidak's post-test was applied. Two-way RM ANOVA indicates whether there was an overall difference between both groups whereas Sidak's post-test compares means at each time point (+2, 3, 4 and 24 h).

### 1.7.3 Time Course Analysis of Ibuprofen or the Compound of Formula (IA) Effects

To analyze substance effects on CYP-induced acute cystitis, individual pairwise comparison between Ibuprofen/CYP or the compound of formula (IA)/CYP and Vehicle/CYP groups.

Two-way RM ANOVA with Sidak's post-test was used. Two-way RM ANOVA indicates whether there was an overall difference between both groups whereas Sidak's post-test compares means at each time point (+2, 3, 4 and 24 h).

It is of note that when individual pairwise comparison was made, statistical significance was indicated by the symbol “*” and non-significance by “^{ns}”. For grouped comparison, non-statistical significance was indicated by the symbol “NS”.

### 1.8 Results

1.8.1 Basal Responses were Similar within the Various Experimental Groups

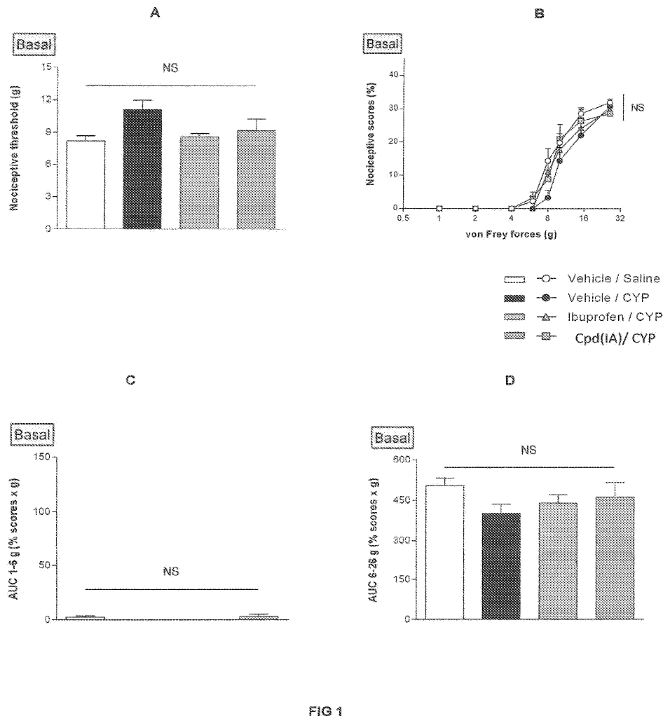

Basal (before CYP or saline injection) nociceptive responses were similar between all experimental groups. Indeed, before CYP or saline injection, no significant difference for any nociceptive parameters was observed between all experimental groups (p>0.05, FIG. 1A-D).

1.8.2 CYP (150 mg/kg, i.p.) Induced Visceral Pain Up to 24 h Post-Injection

In order to confirm CYP-induced visceral pain, comparison of nociceptive response between saline- or CYP-injected rats within the Vehicle groups was performed.

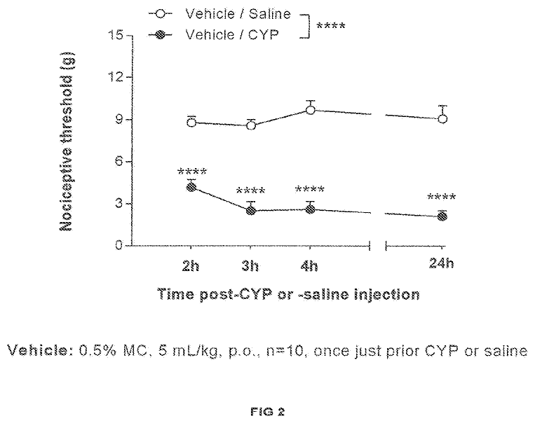

At all evaluated time points (i.e. from 2 h to 24 h), we observed that CYP induced a significant decrease of nociceptive threshold as compared to saline (p<0.0001, FIG. 2). The shape of the time course curve showed that magnitude of CYP effect was lower at 2 h whereas it remained similar from +3 to +24 h.

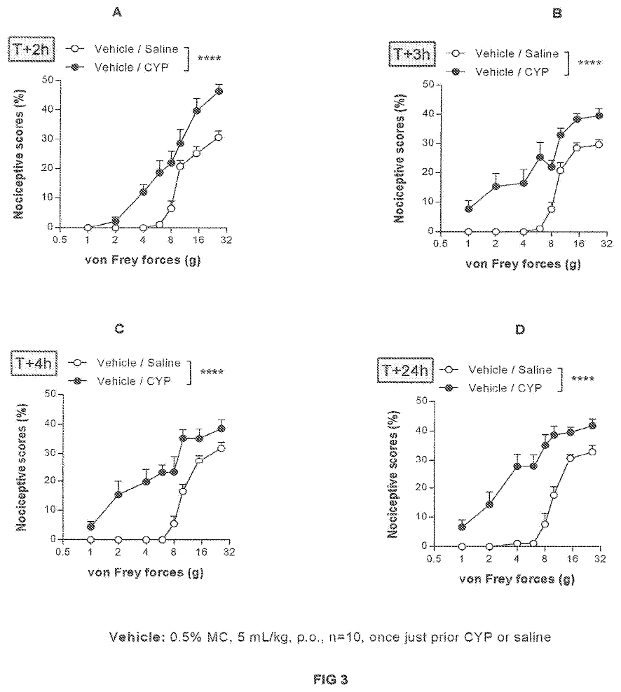

It was further observed that CYP elicited a significant increase in the nociceptive scores at all evaluated time points as compared to saline (p<0.0001, FIG. 3A-D).

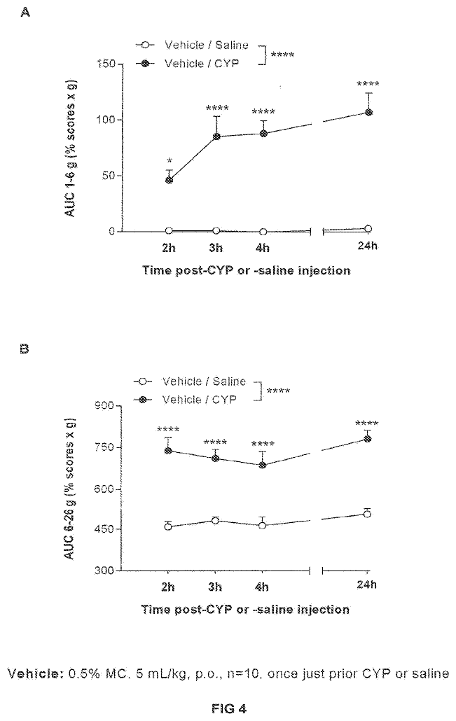

Effects of CYP on nociceptive scores was paralleled with a significant overall increase of AUC 1-6 g (p<0.0001, FIG. 4A). Consistent with results above, effects of CYP were of lower magnitude at +2 h and remained similar from +3 to +24 hours as compared to saline-injected rats (p<0.05 and p<0.0001 at +2 and at +3 to +24 h, respectively, FIG. 4A). Finally, within the Vehicle group, CYP-injected rats exhibited a significant increase of AUC 6-26 g at all time points (p<0.0001, FIG. 4B).

1.8.3 Ibuprofen (300 mg/kg. p.o.) Alleviated CYP-Induced Visceral Pain

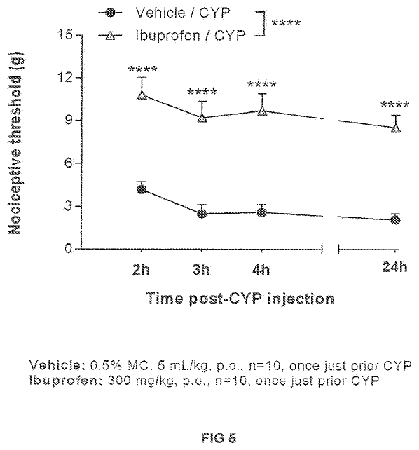

After CYP injection, ibuprofen-treated rats displayed a significant overall increase in nociceptive threshold as compared to vehicle (p<0.0001, FIG. 5). Post hoc statistical analysis of means at each time point showed that levels of significance were maximal for all evaluated time points although a slight and gradual decrease of ibuprofen effect was observed over time (p<0.0001, FIG. 5).

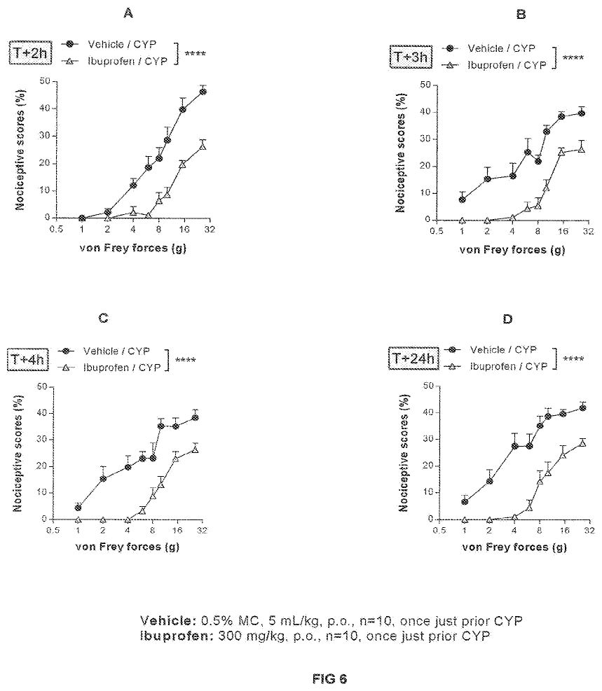

In addition, ibuprofen significantly decreased nociceptive scores all along the observation period (p<0.0001, FIG. 6A-D).

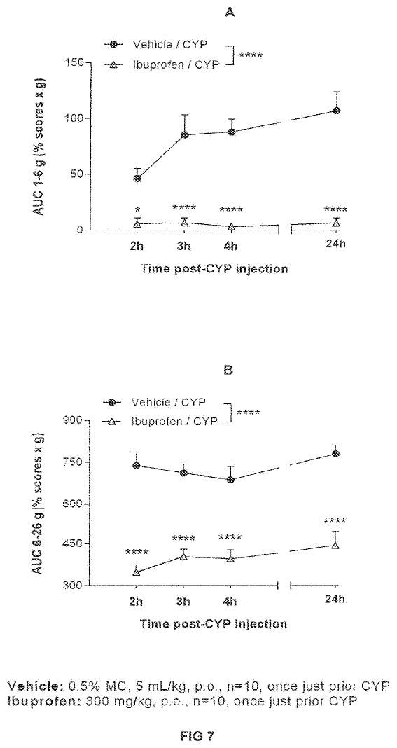

Ibuprofen treatment led to an overall decrease in corresponding AUC 1-6 g (p<0.0001, FIG. 7A). When means were statistically compared at each time point (i.e. post hoc test), we obtained a lower level of significance at +2 h as compared to others time points (p<0.05 vs p<0.0001 at +2 and at +3 to +24 h, respectively, FIG. 7A) although values per se were in the same range (5.5±5.5 and 6.6±4.4 at +2 and +3 h, respectively). This can be explained by the effect of CYP which was of lower magnitude at +2 h within the Vehicle group (FIG. 4A).

FIG. 7B showed that ibuprofen elicited a significant overall decrease in AUC 6-26 g as compared to vehicle (p<0.0001, FIG. 7B). Ibuprofen effect slightly and gradually decreased over time but significance reached the maximum level at all time points (p<0.0001, FIG. 7B).

1.8.4 the Compound of Formula (IA) Alleviated (30 mg/kg, p.o.) Reversed CYP-Induced Visceral Pain

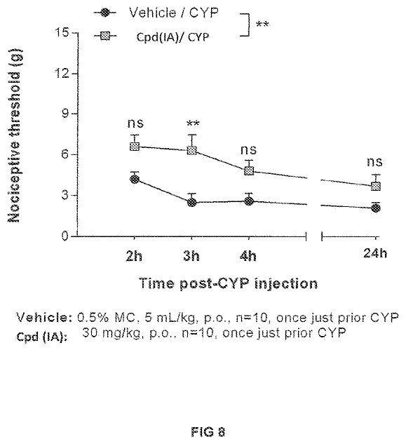

In CYP-injected rats, an overall inhibitory effect of compound of formula (IA) on nociceptive threshold was observed when compared to vehicle (p>0.01, FIG. 8). Comparison at specific time point (i.e. post hoc analysis) showed that statistical significance level was only reached at +3 h (p>0.01, FIG. 8). However, it is noteworthy that values of compound of formula (IA) at +2 and +3 h were in a similar range (6.6±0.8 vs. 6.3±1.2 at +2 and +3 h, respectively). Thus, lack of significance at +2 h could be explained by the lowest magnitude of CYP effect at +2 h (see § 4.2). After +3 h, effect of compound of formula (IA) gradually decreased over time (4.8±0.8 and 3.7±0.8 at +4 and +24 h, respectively).

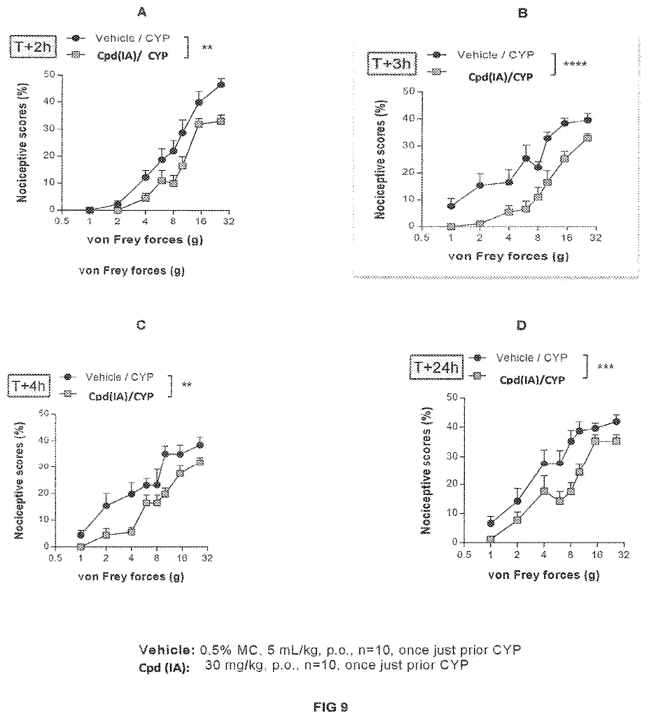

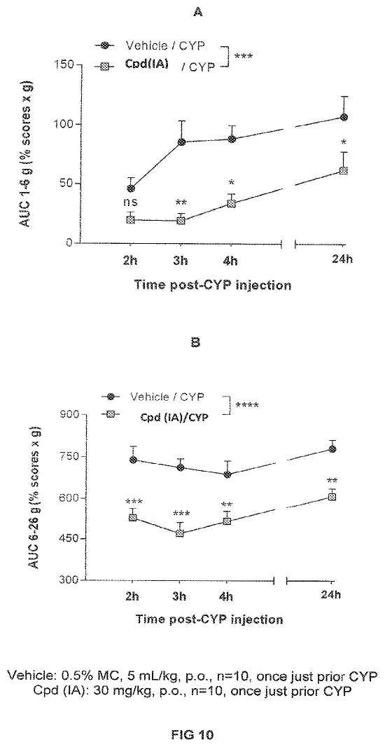

As compared to Vehicle, significant decrease in nociceptive scores was observed in the compound of formula (IA) group starting from 2 h and up to 24 h after CYP injection (p<0.01, FIG. 9). The highest level of significance was obtained at +3 h (p<0.001, FIG. 9B). Decrease in nociceptive scores by the compound of formula (IA) treatment went along with an overall decrease in AUC 1-6 g (p<0.001, FIG. 10A). Consistent with nociceptive threshold results, highest level of significance was reached at +3 h (p<0.01, FIG. 10A) although values per se were in a similar range at +2 and +3 h (19.8±6.9 and 19.3±6.3 at +2 and +3 h, respectively). After 3 h, efficiency of the compound of formula (IA) decreased slightly and gradually over time (34.1±7.9 and 61.8±15.7 at +4 and +24 h, respectively).

Finally, the compound of formula (IA) elicited a significant overall inhibitory effect on CYP-induced AUC 6-26 g increase (p<0.0001, FIG. 10B). Statistical significance was achieved at all time points with a higher level at early time points (p<0.001 at +2 and +3 h, and p, 0.01 at +4 and +24 h, FIG. 10B).

### Example 2: Pharmacokinetic Studies on the Compound of formula (IA)

The urine concentration was evaluated in humans (9 total) for up to 48 hours after administration of a single oral dose of the compound of formula (IA) in the form of a methyl cellulose suspension. Urine samples (pooled serial samples of all urine voided) for determining concentrations of the compound of formula (IA) were collected for individual subjects at the following time intervals: 0 to 8 hours, 8 to 16 hours, 16 to 24 hours, 24 to 32 hours and 40 to 48 hours after administration. Individual subject and summaries of the urine concentration for the compound of formula (IA) by treatment and time interval are provided in Table 4, below.

TABLE 4
Individual subject and summaries of the urine concentration for the compound
of formula (IA) (also referred to as “compound 1a”)

| | | TREATMENT/CONCENTRATION (nM) | | | | | | | |
|---|---|---|---|---|---|---|---|---|---|
| | | Compound | | Compound | | Compound | | Compound | |
| NOM | | 1a 0.2 mg | | 1a 0.6 mg | | 1a 2 mg | | 1a 10 mg | |
| TIME | Variable | Mean | SD | Mean | SD | Mean | SD | Mean | SD |
| 0 to 8 h | URINE CONC | 2311 | 1736 | 1641 | 1196 | 10809 | 6322 | 30964 | 19832 |
| 8 to 16 h | URINE CONC | 394 | 226 | 531 | 363 | 1440 | 768 | 14181 | 8646 |
| 16 to 24 h | URINE CONC | BQL | BQL | BQL | BQL | 400 | 280 | 1994 | 905 |
| 24 to 32 h | URINE CONC | BQL | BQL | BQL | BQL | BQL | BQL | 535 | 55 |
| 32 to 40 h | URINE CONC | BQL | BQL | BQL | BQL | BQL | BQL | 240 | BQL |
| 40 to 48 h | URINE CONC | BQL | BQL | BQL | BQL | BQL | BQL | BQL | BQL |

In vitro activity against human nociception receptors; Ki 2.45 nM, EC 50 4.0 nM

The data in Table 4 shows the mean urine concentration levels of the compound of formula (IA) at particular time intervals and standard deviations after administration of particular doses (0.2 mg, 0.6 mg, 2 mg and 10 mg) of the compound of formula (IA). Table 4 shows that even at the lowest dose of the compound of formula (IA) (0.2 mg), the urine concentration is orders of magnitude greater than the in vitro activity of the compound of formula (IA) was measured by Ki and EC₅₀.

### Example 3: A Phase 1b Blinded, Placebo-Controlled, Crossover Study to Investigate Effects of the Compound of Formula (IA) in Female Human Subjects with Interstitial Cystitis/Bladder Pain Syndrome

### Primary Assessments:

To assess the effects of the compound of formula (IA) on change in mean average daily bladder pain/discomfort symptom score in subjects diagnosed with IC/BPS, as compared to placebo. Each subject is required to identify their most bothersome bladder pain/discomfort symptom at screening. To be eligible for randomization, the subject must report experiencing this specific symptom during a 2-week single-blind placebo run-in period, at a sufficient severity (a protocol-specified intensity or frequency).

The average daily bladder pain/discomfort symptom score is assessed on an 11-point numeric rating scale (NRS) twice daily (morning and evening) using an e-diary. The 11-point NRS ranges from 0=“no bladder pain/discomfort” to 10=“worst pain pain/discomfort you can imagine”. The symptom considered most bothersome to subject is based on a consensus decision between subject and primary investigator done in a protocol-specified IC/BPS-related symptom assessment procedure during screening (and in the e-diary).

### Secondary Assessments:

To assess the effects of the compound of formula (IA) on change in mean number micturitions per 24 hours in subjects diagnosed with IC/BPS, as compared to placebo. The subject is asked to record all micturition episodes (acts of urinating) in an e-diary over the 7 days preceding each scheduled investigative site clinic visit. The subject is required to record the time, type, and urgency of each micturition episode on all 7-days, and additionally record urine volume over the 2-day weekend.
Episode type with be categorized as urinated (passed urine in toilet) or incontinence (involuntary release of urine).
Episode urgency (the sensation of a sudden compelling desire to urinate that is difficult to defer) is rated on an 11-point NRS for each episode; and the NRS ranges from 0=“no urgency” to 10=“urgency as bad as you can imagine”.
Episode volume is total urine volume per voiding during a single recorded episode.

### Other Assessments:

To assess the effects of the compound of formula (IA) on changes in other bladder pain/discomfort components and additional lower urinary tract components.

The following measures are done at home, by subject in the e-diary.
Average daily and maximum (worst) daily bladder pain/discomfort symptom score obtained twice daily (morning and evening).
All micturition episodes (acts of urinating) occurring during the week (7-days) preceding each scheduled investigative site clinic visit. The time, type, intensity, and volume of each episode are collected over the 24-hour period of each day.

The following measures are performed at clinic visits:
Bladder Pain/Interstitial Cystitis Symptom Score (BPIC-SS). The BPIC-SS contains 8 questions concerning bladder pain over the previous 7 days. Question 1-5 assess how often subject urinated because of pain, needed to urinate just after a previous urination, urinated to avoid pain, had pressure in their bladder, and had pain in their bladder, are rated on a 5-point scale from 0= “not at all” to 4=“a great deal”. Question 6 and 7 assess how bothered was subject by frequent urination during daytime and during the night, rated on a 5-point scale from 0=“not at all” to 4=“a great deal”. Question 8 assesses subject's worst bladder pain in past 7 days, rated on an 11-point NRS with range 0=“no bladder pain” to 10=“worst possible bladder pain”. The BPIC-SS total score is the sum of the individual question scores and range from 0 to 38 with higher scores indicating a worse situation; a score of ≥19 may represent moderate/severe disease activity. Total score and worst bladder pain (Q8) is to be evaluated.
O'Leary-Sant Interstitial Cystitis Symptom Index (ICSI). The ICSI contains 4 questions that measure urgency and frequency of urination, night time urination, and pain or burning. Each question is rated using a 6-point scale that ranges from 0=no symptoms/problems to 5=worst symptoms/problems. Total scores range from 0 to 20. A 1-week recall period will be used.
O'Leary-Sant Interstitial Cystitis Problem Index (ICPI). The ICPI contains 4 questions that assesses severity of symptoms (frequency, nocturia, urgency, discomfort) and how much of a problem the symptoms cause for the patient. Each question is rated using a 5-point scale that ranges from 0=“no problem” to 4=big problem”. Total scores range from 0 to 16. A 1-week recall period will be used.
Subject Global Response Assessment (SGRA). The SGRA is a self-rated balanced measure of subject's clinical condition relative to baseline. There is 1 question: a) “Compared to when you started the study, how would you rate your overall interstitial cystitis/bladder pain symptoms now? A 7-point rating scale is used: 1=“markedly worse”, 2=“moderately worse”, 3=“slightly worse”, 4=“no change”, 5=“slightly improved”, 6=“moderately improved” or 7=markedly improved”.
Subject Global Response Assessment (SGRA)-bothersome symptoms. The SGRA is used to evaluate a subject's clinical condition of most bothersome symptoms relative to baseline and contains 2 questions: a) “Compared to when you started the study, how would you rate the your most bothersome bladder pain/discomfort symptom now?” and b) “Compared to when you started the study, how would you rate your most bothersome lower urinary tract symptom now?” For both questions, a 7-point rating scale is used: 1=“markedly worse”, 2=“moderately worse”, 3=“slightly worse”, 4=“no change”, 5=“slightly improved”, 6=“moderately improved” or 7=“markedly improved”.
Nocturia Sleep Quality Scale (NSQS). The NSQS consists of six items assessing hypothesized domains of number of night-time awakenings, sleep quantity, and sleep quality. The NSQS employs a short recall period of “from the time you went to bed, with the intention to sleep, until you woke up to start your day” to allow patients to assess the impact of nocturia on sleep over time.

### Safety Assessments:

To assess safety and tolerability of the Compound of formula (IA) as measured by adverse events (AEs), chemistry, hematology, urinalysis (including microscopy) results, electrocardiograms (ECGs), physical examination, vital signs, Columbia-Suicide Severity Rating Scale (C-SSRS), Hospital Anxiety and Depression Scale (HADS), Karolinska Sleepiness Scale (KSS), and Digit Symbol Substitution Test (DSST).

### Diagnosis and Main Criteria for Inclusion:

The following inclusion/exclusion criteria are used at screening visit 1 to determine eligibility for entry into Run-in Phase. Enrollment Criteria into the Double-blind Randomized Treatment Period are also provided below.

### Inclusion:

1. Non-pregnant (confirmed by urine pregnancy test), non-lactating female, age 18-64 years, capable of voiding independently.
2. Have diagnosis of interstitial cystitis/bladder pain syndrome (IC/BPS) in accordance with American Urological Association (AUA) Guidelines that was documented in the subject's medical records at least 6 months prior to screening.
3. Have bladder pain/discomfort with accompanying lower urinary tract symptoms that meet the following criteria at screening as evaluated by a health care provider:
   a. Subject has complaint of bladder pain/pressure/discomfort for ≥6 months accompanied by other lower urinary tract symptoms for ≥6 months
   b. Subject has Bladder Pain/Interstitial Cystitis Symptom Scale (BPIC-SS) total score of ≥19
   c. Subject average daily most bothersome bladder pain during past week is ≥4 and ≤8 as rated by subject using 11-point numeric rating scale of range 0 (no pain) to 10 (worst pain)
   d. Subject micturition frequency is estimated to be ≥11 and ≤30 voids per 24 hours in last week.
4. Has cystoscopy findings at screening, or obtained within 6 months of screening, that exclude other conditions that cause bladder pain discomfort (e.g. transitional cell carcinoma of the bladder, endometriosis of the bladder).
5. Did not receive pharmacologic treatment for IC/BPS in past 30 days; or, did receive oral pharmacologic treatment for interstitial cystitis/bladder pain syndrome at a stable dose/regimen during past 30 days, and in the judgement of investigator, is expected to remain on the same stable dose/regimen throughout the study.
6. Meet study inclusion criteria despite history of previous or continuing treatments including conservative treatment, which may include one or more of the following: a) timed voiding and behavioral modification therapy, b) dietary restrictions, c) stress reduction, and despite oral therapy with any of the following medications: antidepressants, antihistamines, antimuscarinic and anticholinergic agents, alpha adrenergic blockers, analgesics, and pentosan polysulfate.
7. Be considered, in the opinion of the clinical site investigator, to be generally healthy based on the results of a medical history, physical examination, 12-lead ECG, and laboratory profile
8. Be able to speak, read, write, and understand English, understand the consent form, and effectively communicate with the study staff.
9. Voluntarily provide written informed consent and be willing and able to complete all study procedures including a daily Bladder Diary (pain and void assessments)

### Exclusion:

1. Has pelvic floor pain (score >5 out of 10 on an 11-point NRS pain scale)
2. Has been treated with any opioid therapy for any indication in past 30 days
3. Has used >70 oral milligram morphine equivalents of opioids per week to control IC/BPS pain within past 30 days or is expected to require this level of opioid pain control during study.
4. Had urinary tract infection (UTI), including bacterial cystitis, within the past 30 days, or has a history of recurrent UTI as defined as 3 or more episodes in the last 6 months. Note: subjects with infection may be rescreened in 30 days.
5. Hematuria on microscopic examination. Note: subjects with micro-hematuria may be approved for enrollment following discussion and approval with study medical monitor.
6. Had surgical procedure at any time that affected bladder function (e.g. augmentation cystoplasty, cystectomy, cystolysis, neurectomy, hypogastric nerve plexus ablation) or received a botulinum toxin bladder injection within previous 9 months.
7. Received intravesical therapy or bladder hydrodistention (instillations that included liquid or drug delivery devices, pentosan polysulfate sodium, lidocaine, steroid, heparin, chondroitin and any combination or additional formulation) within past 30 days.
8. Used a sacral and/or pudendal nerve neuromodulation device (e.g. Interstim) within last 6 months (not excluded if device started >6 months ago and subject on stable setting for past 3 months).
9. Had percutaneous Tibial Nerve Stimulation (PTNS) treatment within past 90 days
10. Has concurrent malignancy or history of cancer, except noninvasive skin cancer, within the last 5 years (note: patients with history of cancer are eligible if the they had undergone therapy and were considered disease free for at least 5 years).
11. Had bladder tumor (benign or malignant) at any time.
12. Has active genital herpes or vaginitis
13. Has a urethral diverticulum
14. Had cyclophosphamide or chemical cystitis, or tuberculosis, or pelvic radiation
15. Has uncontrolled diabetes
16. Currently pregnant or breastfeeding, or plan to become pregnant during course of study
17. Has moderate to severe hepatic impairment defined as Child-Pugh Class B or C.
18. Has any history and/or current evidence of other medical (e.g., cardiac, respiratory, gastrointestinal, renal, malignancy other than basal cell carcinoma), neurological, or psychiatric conditions that, in the opinion of the investigator, could affect the subject's safety or interfere with the study assessments.
19. Has clinically significant depression based on a score of ≥11 on the Hospital Anxiety and Depression Scale (HADS); suicidal ideation associated with actual intent and a method or plan in the past year (“Yes” answers on items 4 or 5 of the Columbia-Suicide Severity Rating Scale [C-SSRS]); a previous history of suicidal behaviors in the past 5 years (“Yes” answer to any of the suicidal behavior items of the C-SSRS); or any lifetime history of serious or recurrent suicidal behavior. Non-suicidal, self-injurious behavior is not a trigger for a risk assessment unless indicated as per the judgment of the clinical site investigator
20. Has history of or any current conditions that might interfere with drug absorption, distribution, metabolism, or excretion (including any surgical interventions for weight loss).
21. Has a substance use disorder or has a positive UDT for illicit drugs (including tetrahydrocannabinol [THC]), non-prescribed controlled substances (opioid or non-opioid), or alcohol at Screening or has ever had an opioid overdose for any reason.
22. Has a body mass index <20 kg/m² or >35 kg/m²
23. Has abnormal kidney function as evidenced in screening by abnormal blood urea nitrogen and creatinine values and an estimated glomerular filtration rate (eGFR)<60 mL/min/1.73 m^{2‘.}
24. Has a clinically significant abnormality in clinical chemistry, hematology, or urinalysis, including serum glutamic-oxaloacetic transaminase/aspartate aminotransferase or serum glutamic pyruvic transaminase/alanine aminotransferase >3-fold the upper limit of the reference range, or serum creatinine >2 mg/dL at Screening.
25. Has abnormal cardiac conditions including uncontrolled hypertension (>140 mm Hg systolic/90 mm Hg diastolic) or QTcF >450 msec (male) and QTcF >470 msec (female) confirmed on a repeat ECG or a clinically significant abnormal ECG.
26. Has excessive caffeine consumption ≥300 mg/day (e.g., approximately three 6-oz cups of caffeinated coffee, or three 12-oz caffeinated sodas, or three 8-oz caffeinated tea beverages).
27. Was dosed in a clinical drug study during the 30 days (or 5 half-lives of investigational drug treatment, whichever is longer) preceding the initial dose of study drug dispensed in this study.
28. Was previously exposed to the Compound of formula (IA).
29. Is scheduled for surgery during the study.

### Randomization Criteria (Double-Blind Phase):

Subjects must meet the following criteria for randomization into the Double-blind Randomized Treatment Phase following the completion of daily Bladder Diary:
a) average daily bladder pain score is ≥5 for at least 9 days of the run-in phase
b) micturition episodes average is ≥10 and <30 in each 24-hour period for at least 3 days
c) Bladder Pain/Interstitial Cystitis Symptom Scale (BPIC-SS) score is ≥19
d) incoming IC/BPS therapy was stable dose/regime and expected to remain so during study
e) diary was completed by subject appropriately, according to investigator instruction.

### Study Design:

Phase 1b, blinded, placebo-controlled crossover study assessing the effects of oral administration of 1 mg/day of the Compound of Formula (IA) in female subjects with IC/BPS, as compared to placebo.

This study consists of 2 phases: Pre-randomization Phase consisting of a Screening Period (up to 4 weeks) and Single-blind Placebo Run-in Period (2 weeks), and a Randomized Treatment Phase consisting of a Double-blind Treatment Period (9 weeks) and Follow-up Period (up to 1 week).

TABLE 5
Study Scheme

| | Phase | | | | | | | | | | | | |
|---|---|---|---|---|---|---|---|---|---|---|---|---|---|
| | Pre-randomization | | | | | | | | | | | | |
| | Screen | Single-blind Run- | | Randomized Treatment | | | | | | | | | |
| Period | [washout] | in [enrichment] | | Double-blind Treatment | | | | | | | | | Follow-up |
| Week | Up to 4 | −2 | −1 | 1 | 2 | 3 | 4 | 5 | 6 | 7 | 8 | 9 | 10 |
| Day | −42 to −15 | −14 | −1 | 1 | 14 | | 28 | | 42 | | 56 | 63 | 70 |
| Visit | V1 | | V2 | | V3 | | V4 | | V5 | | V6 | V7 | V8 |

| | | | | | | |
|---|---|---|---|---|---|---|
| Treatment* | placebo | placebo | active | active | active | placebo |

*Final protocol will not disclose treatment regimen to subject or investigator.

Screening/washout Period (Days −42 to −15): Informed consent is obtained from each subject before she submits to any study procedures performed in this study. The assessment of study eligibility criteria is initiated at the screening visit and includes medical history, physical examination, vital signs, laboratory results, urine culture, pregnancy test, and drug screen. If a washout of prohibited medications (Appendix A) is required, this washout should be completed during screening (Days −42 to −15). The subject must have diagnosis of interstitial cystitis/bladder pain syndrome (IC/BPS) in accordance with American Urological Association (AUA) Guidelines that was documented in the subject's medical records at least 6 months prior to screening. The subject must have a cystoscopy at screening, if not obtained within the preceding 6 months to exclude other conditions and document the presence or absence of a Hunner lesion. The subject should either have received no pharmacologic treatment for IC/BPS in past 30 days; or, have received oral pharmacologic treatment for IC/BPS at a stable dose/regimen during past 30 days and in the judgement of investigator, is expected to remain on the same stable dose/regimen throughout the study. Subjects with opioid use for any reason in past 30 days, or expected to require opioid therapy for bladder pain, or any indication, are excluded from enrollment in the study. The subject identifies their most bothersome bladder pain/discomfort symptom and their most bothersome lower urinary tract symptoms related to IC/BPS.

Single-blind Placebo Run-in Period (Days −14 to −1): Once a subject successfully completes the screening visit, he/she enters the placebo run-in period and ingest 1 tablet of study-drug each evening 30 minutes before bedtime. Dosing is entered each night in an e-diary. The subject records their average daily and worst daily bladder pain/discomfort score twice daily in morning and night. The subject records all micturition episodes over a 24-hour period (including time, type, intensity, and volume of each) during the week that precedes visit 2. The subject must meet the following criteria to be eligible for enrollment in the double-blind treatment period.
a) average daily bladder pain/discomfort score is ≥5 for at least 9 days of the run-in phase
b) micturition episodes average is ≥10 and <30 in each 24-hour period for at least 3 days
c) Bladder Pain/Interstitial Cystitis Symptom Scale (BPIC-SS) score is ≥19
d) incoming IC/BPS therapy was stable dose/regime and expected to remain so during study
e) diary was completed by subject appropriately, according to investigator instruction.

If the subject has met enrollment criteria and completed all assessments, they are randomized.

### Treatment Period (Days 1 to 63)

All subjects are assigned to the same crossover treatment regimen of the Compound of Formula (IA) and placebo. The subject attends clinic visits every 2 weeks, with a virtual/phone call conducted during the weeks that a clinic visit is not scheduled.

The subject continues to record their bladder pain scores twice daily. The subject also records all micturition episodes over a 24-hour period during the 7-day period prior to each scheduled clinic visit to investigational site. At clinic visits, the subject completes the efficacy assessments (BPIC-SS, ICSI, ICPI, NSQS, GRA, and SGRAB) and safety/other assessments.

### End of Study (EOS) (Day 63/Early Termination [ET1]):

Subjects undergo EOS procedures at either the end of double-blind treatment, or upon early discontinuation from the study. If assessments called for at EOS were performed on the same day of study completion or on the same day as ED and no study drug was administered after those assessments, those assessments need not be repeated.

### Follow-up Period (Days 64-70)

A follow-up phone call will be completed 7 to 10 days after the last dose of study drug to monitor adverse events and use of concomitant medication/therapy since the previous visit.

While the subject matter of this disclosure has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

### REFERENCES

Augé C. et al., 2011. Pharmacological modulation of urinary bladder inflammation in a rat model of cyclophosphamide-induced acute bladder pain. Inflamm. Res. 60 (Suppl 1): S248.
Augé C. et al., 2013. Relevance of the cyclophosphamide-induced cystitis model for pharmacological studies targeting inflammation and pain of the bladder. Eur. J. Pharmacol. 707(1-3):32-40.
Boucher M. et al., 2000. Cyclophospham ide-induced cystitis in freely-moving conscious rats: behavioral approach to a new model of visceral pain. J. Urol. 164(1):203-8.
Cox P J., 1979. Cyclophosphamide cystitis-identification of acrolein as the causative agent. Biochem. Pharmacol. 28(13):2045-9.
Juszczak K. et al., 2007. Animal models of overactive bladder: cyclophosphamide (CYP)-induced cystitis in rats. Folia Med. Cracov. 48(1-4):113-23.
Lluel P. et al., 2010. Cyclophosphamide-induced cystitis in conscious female rats: development and pharmacological validation of an experimental model of referred visceral pain. Annual meeting of the International Continence Society (ICS), Toronto, Canada.
Payne C K. et al., 2007. Interstitial cystitis and painful bladder syndrome. J. Urol. 177(6):2042-9.
Smaldone M C. et al., 2009. Multiplex analysis of urinary cytokine levels in rat model of cyclophosphamide-induced cystitis. Urology. 73(2):421-6.
Takagi-Matsumoto H. et al., 2004. Effects of NSAIDs on bladder function in normal and cystitis rats: a comparison study of aspirin, indomethacin, and ketoprofen. J. Pharmacol. Sci., 95, 458-465.
Bosch P C. Examination of the Significant Placebo Effect in the Treatment of Interstitial Cystitis/Bladder Pain Syndrome. Urology 2014; 84(2):321-325.
Garzon S, Lagana A S, Casarin J et al. An update on treatment options for interstitial cystitis. Menopause Rev 2020; 19(1): 35-43.
Hanno M P, Burks D A, Clemens J Q, et al. American Urological Association (AUA) Guideline: Diagnosis and treatment of interstitial cystitis/bladder pain syndrome 2014.
FDA 2019 Draft Guidance Document. Interstitial Cystitis/Bladder Pain Syndrome (IC/BPS): Establishing Effectiveness of Drugs for Treatment Guidance for Industry.

1. A method of treating interstitial cystitis/bladder pain syndrome in a human subject in need of such treatment, comprising administering to the human subject a therapeutically effective amount of a compound having the formula (I): 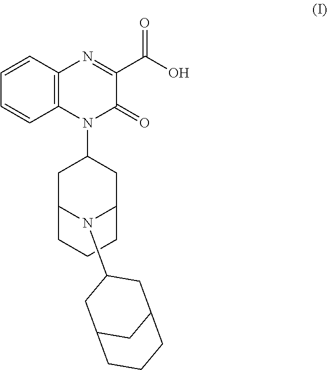 [SMILES: O=C(O)c1nc2ccccc2n(C2CC3CCCC(C2)N3C2CC3CCCC(C3)C2)c1=O]
or a pharmaceutically acceptable salt thereof.

2. A method of treating or relieving a symptom associated with interstitial cystitis/bladder pain syndrome in a human subject in need of such treatment, comprising administering to the subject a therapeutically effective amount of a compound having the formula (I): 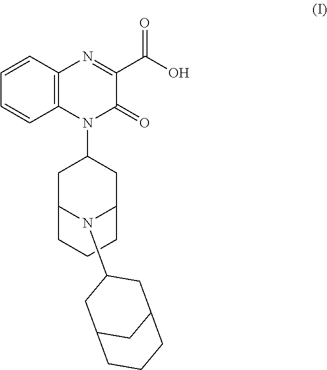 [SMILES: O=C(O)c1nc2ccccc2n(C2CC3CCCC(C2)N3C2CC3CCCC(C3)C2)c1=O]
or a pharmaceutically acceptable salt thereof.

3. The method of claim 2, wherein the symptom is visceral pain.

4. The method of claim 2, wherein the symptom is urinary urgency.

5. The method of any one of claims 1-4, where the compound of formula (I) or a pharmaceutically acceptable salt thereof is the compound of formula (I′): 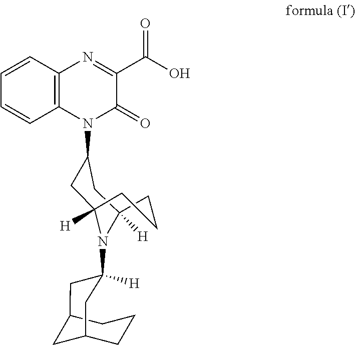
or a pharmaceutically acceptable salt thereof.

6. The method of any one of claims 1-5, wherein the method comprises administering to the subject a therapeutically effective amount of a pharmaceutically acceptable salt of the compound.

7. The method of claim 6, wherein the pharmaceutically acceptable salt is ap-toluenesulfonic acid salt, a sulfate salt, a phosphoric acid salt or a hydrochloride salt.

8. The method of claim 6 or 7, wherein the pharmaceutically acceptable salt is ap-toluenesulfonic acid salt.

9. The method of any one of claims 1-8, wherein the compound or a pharmaceutically acceptable salt thereof is administered orally, parenterally, intravenously, intramuscularly, buccally, or transdermally.

10. The method of claim 9, wherein the compound or a pharmaceutically acceptable salt thereof is administered orally.

11. The method of any one of claims 1-10, wherein the therapeutically effective amount of the compound or a pharmaceutically acceptable salt thereof is from about 0.001 mg to about 30 mg.

12. The method of any one of claims 1-11, wherein the therapeutically effective amount of the compound or a pharmaceutically acceptable salt thereof is from about 0.005 mg to about 25 mg.

13. The method of any one of claims 1-12, wherein the therapeutically effective amount of the compound or a pharmaceutically acceptable salt thereof is from about 0.075 mg to about 12 mg.

14. The method of any one of claims 1-13, wherein the therapeutically effective amount of the compound or a pharmaceutically acceptable salt thereof is from about 0.1 mg to about 10 mg.

15. The method of any one of claims 1-14, wherein the compound or a pharmaceutically acceptable salt thereof, is administered once daily.

16. The method of any one of claims 1-15, wherein the compound or a pharmaceutically acceptable salt thereof, is administered at night.

17. The method of claim 16, wherein the compound or a pharmaceutically acceptable salt thereof, is administered at night prior to bedtime of the human subject.

18. The method of any one of any one of claims 1-17, wherein the compound or a pharmaceutically acceptable salt thereof, is administered twice daily.

19. The method of claim 18, wherein a therapeutically effective amount of the compound or a pharmaceutically acceptable salt thereof, is administered approximately every 12 hours.

20. The method of claim 18 or claim 19, wherein the method comprises administering a first therapeutically effective amount of the compound or a pharmaceutically acceptable salt thereof during daytime, and administering a second therapeutically effective amount at night prior to bedtime of the human subject.

21. The method of any one of claims 18-20, wherein the method comprises administering a same therapeutically effective amount of the compound or a pharmaceutically acceptable salt thereof each time.

22. The method of any one of claims 18-21, wherein the method comprises administering two different therapeutically effective amounts of the compound or a pharmaceutically acceptable salt thereof during the daily administrations.

23. The method of claim 22, wherein the second therapeutically effective amount is about 2-fold greater than the first therapeutically effective amount.

24. The method of claim 22, wherein the second therapeutically effective amount is about 5-fold greater than the first therapeutically effective amount.

25. The method of claim 22, wherein the second therapeutically effective amount is about 10-fold greater than the first therapeutically effective amount.

26. The method of claim 22, wherein the second therapeutically effective amount is about 25-fold greater than the first therapeutically effective amount.

27. The method of claim 22, wherein the second therapeutically effective amount is about 100-fold greater than the first therapeutically effective amount.

28. The method of any one of claims 1-27, wherein the administration of the compound or a pharmaceutically acceptable salt thereof increases the pressure threshold for micturition in the human subject by from about 30% to 80%.

29. The methods of any one of claims 1-28, wherein administration of the compound of formula (I) does not affect blood pressure of the human subject.

30. The method of any one of claims 1-29, wherein the compound or a pharmaceutically acceptable salt thereof, is administered in a pharmaceutically acceptable composition.

31. The method of any one of claims 1-29, further comprising administering an antimuscarinic agent to the human subject.

32. The method of claim 31, wherein the antimuscarinic agent is selected from the group of oxybutynin, tolterodine, trospium, solifenacin and darifenacin, or a pharmaceutically acceptable salt thereof.

33. The method of any one of claims 1-32, wherein the method comprises administering a therapeutically effective amount of a compound of formula (IA):

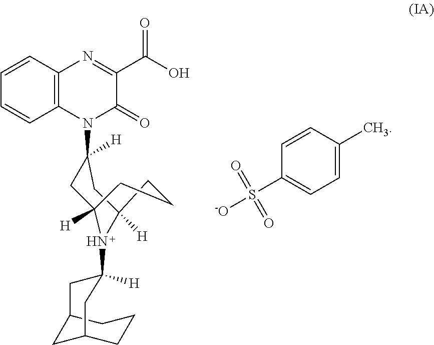

34. A method of treating interstitial cystitis/bladder pain syndrome in a human subject in need of such treatment, comprising administering to the human subject a therapeutically effective dose of a compound of formula (I′): 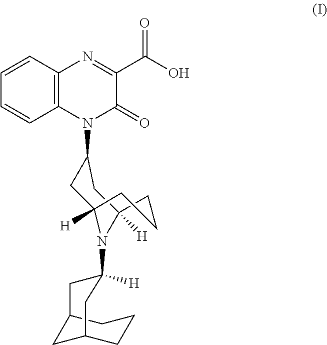
or a pharmaceutically acceptable salt thereof, wherein the human subject further suffers from a sleep disorder.

35. The method of claim 34, wherein the sleep disorder is insomnia associated with alcohol cessation.

36. The method of claim 34 or 35, wherein the human subject is a female of 50 years of age or older.

37. A method of treating insomnia in a human subject in need thereof, comprising administering to the human subject a therapeutically effective amount of a compound of formula (I′): 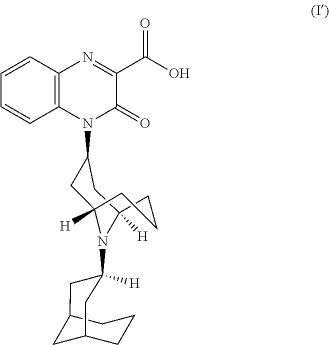
or a pharmaceutically acceptable salt thereof, wherein said human subject further suffers from interstitial cystitis/bladder pain syndrome.

38. The method of claim 37, wherein the method comprises administering to the human subject a therapeutically effective amount of the compound of formula (IA): [chemical structure]
